(19) United States
(12) Patent Application Publication
(10) Pub. No.: US 2022/0193321 A1
(43) Pub. Date: Jun. 23, 2022

(54) VASCULAR CONDUIT TO FACILITATE TEMPORARY DIRECT ACCESS OF A VESSEL

(71) Applicant: Silk Road Medical, Inc., Sunnyvale, CA (US)

(72) Inventors: Stewart M. Kume, Sunnyvale, CA (US); Zack Tegels, Sunnyvale, CA (US); Kristen Lipschultz, Sunnyvale, CA (US); Brad Steele, Sunnyvale, CA (US)

(21) Appl. No.: 17/555,127

(22) Filed: Dec. 17, 2021

Related U.S. Application Data
(60) Provisional application No. 63/127,818, filed on Dec. 18, 2020.
(60) Provisional application No. 63/187,759, filed on May 12, 2021.

(51) Int. Cl. A61M 1/36

(52) U.S. Cl. CPC A61M 1/3655

(57) ABSTRACT

Methods, devices, and systems establish and facilitate arterial access for interventional procedures, such as stenting, angioplasty, grafting, replacement valve therapy, and atherectomy.

### CROSS-REFERENCE TO RELATED APPLICATIONS

This application claims priority to co-pending U.S. Provisional Application Ser. No. 63/127,818, filed Dec. 18, 2020, and 63/187,759, filed May 12, 2021. The entire contents of each are hereby incorporated by reference herein in their entireties.

### BACKGROUND

Gaining temporary access into vessels such as the common carotid artery (CCA) with an arterial access sheath is a critical aspect of different interventional procedures including Trans-Carotid Artery Revascularization (TCAR) procedures and other procedures in which the arterial access sheath is inserted directly into the CCA. The distance between the clavicle and the carotid bifurcation is preferably at least 5 cm to ensure safe insertion of the arterial sheath into the CCA. This portion of the CCA is also preferably relatively disease-free. Atherosclerotic disease usually consists of deposits of plaque P that in the carotid artery narrow the junction between the CCA and the internal carotid artery (ICA), an artery which provides blood flow to the brain. Arterial sheath insertion in this location can liberate embolic material if one or more system components contact the plaque deposits. For example, the introducer guidewire, arterial dilator, or arterial sheath can all cause plaque to break apart leading to embolic particles being released. Embolic particles generated can enter the cerebral vasculature, leading to neurologic consequences such as transient ischemic attacks TIA, ischemic stroke, or death. In addition, should such narrowings become severe, blood flow to the brain is inhibited with serious and sometimes fatal consequences. The shorter the CCA, the greater the risk for this complication.

Additionally, arterial sheath insertion in the CCA can cause arterial dissection, particularly for patients in which the CCA is located relatively deep beneath the skin surface. The insertion angle of the arterial sheath for deep CCA patients also is generally steeper (e.g., 50-80 degrees to horizontal). Steep insertion angles of the arterial sheath and any sheath dilator used to advance the sheath through the vessel wall can also increase the risk of arterial dissection and possibly perforation of the posterior wall of the artery.

Furthermore, some carotid artery patients or procedures (e.g. procedures on short/deep vessels, patients with disease, dissections, patients having scar tissue, etc.) may require a larger bore sheath or delivery system, and thus a larger entry point into the carotid artery. Such patients and procedures would benefit from use of a large bore conduit (e.g. as part of a large bore delivery system). Additionally, the conduits described herein can improve surgical outcomes and reduce patient discomfort. For example, sutures such as purse string sutures can be used to attach the conduit to the access site during left ventricular apical access. Using a conduit, such as the conduits described herein stabilizes access site tissue and thus improves surgical outcomes and reduces patient discomfort.

### SUMMARY

Provided herein are, inter alia, solutions to these and other problems in the art. Aspects of the current subject matter relate inter alia to a system for accessing an artery via a trans-carotid approach. Related systems and methods are also provided.

Consistent with some aspects of the current subject matter, a system for accessing an artery via a trans-carotid approach is disclosed. The system includes an arterial access device comprising a distal connector, a lumen, and a hemostasis valve spaced away from the distal connector by the lumen; and a conduit having an inner lumen extending between a proximal end and a distal end, and a coupler at the proximal end, the distal end of the conduit configured for surgical end-to-side colligation with a vessel, wherein the distal connector of the arterial access device is configured to operatively couple with the coupler at the proximal end of the conduit placing the lumen of the arterial access device in fluid communication with the inner lumen of the conduit.

In variations, one or more of the following features may be included in any feasible combination. For example, in some implementations, the conduit is a flexible, tubular structure formed of a biotextile. In some implementations, the conduit is a vascular graft. In some implementations, the vascular graft is formed of a base material selected from the group consisting of polyethylene terephthalate, polyester, nylon, expanded polytetrafluoroethylene, heparin-bonded ePTFE, hooded PTFE, ring reinforced ePTFE, and nylon-ePTFE woven hybrid. In some implementations, the conduit inner lumen has a diameter that is greater than about 2 mm up to about 10 mm. In some implementations, the conduit inner lumen has a diameter that is between about 6 mm and about 16 mm. In some implementations, the conduit inner lumen has a diameter that is between about 8 mm and about 10 mm. In some implementations, the arterial access device further includes a distal sheath coupled to and extending distal from the distal connector, the distal sheath comprising a sheath lumen. In some implementations, the sheath lumen is sized 7 Fr to 10 Fr. In some implementations, the sheath lumen is sized 12 Fr to 36 Fr. In some implementations, the sheath lumen is sized 14 Fr to 20 Fr. In some implementations, a length of the conduit between the proximal end and the distal end is longer than a length of the distal sheath so that when the distal sheath extends through the inner lumen of the conduit a distal end of the distal sheath remains proximal to the distal end of the conduit and inside the conduit inner lumen. In some implementations, the length of the conduit is 5 cm to 30 cm, and wherein the length of the distal sheath is 4 cm to 29 cm. In some implementations, the vessel is the common carotid artery, femoral artery, radial artery, brachial artery, ulnar artery, or subclavian artery.

In some implementations, the system further includes a shunt fluidly connected to the arterial access device, wherein the shunt provides a pathway for blood to flow from the arterial access device to a return site. In some implementations, the system further includes a flow control assembly coupled to the shunt and adapted to regulate blood flow through the shunt. In some implementations, the system further includes an aspiration device coupled to a port on the shunt. In some implementations, the aspiration device is a syringe or a pump. In some implementations, the system further includes a distal adapter coupled to and extending distal from the distal sheath.

In another, interrelated aspect, a system for accessing an artery via a trans-carotid approach is provided. The system includes a conduit having an inner lumen extending between a proximal end and a distal end, the distal end of the conduit configured for surgical end-to-side colligation with a vessel; a coupler positioned at the proximal end of the conduit; and a shunt fluidly connected to the coupler so that the inner lumen of the conduit is coupled to a lumen of the shunt that provides a pathway for blood to flow from the conduit through the shunt and to a return site.

In variations, one or more of the following features may be included in any feasible combination. For example, in implementations, the system further includes a port fluidly communicating with the shunt, wherein the port is configured to connect an aspiration device to the shunt, wherein the distal connector of the arterial access device is configured to operatively couple with the coupler at the proximal end of the conduit placing the lumen of the arterial access device in fluid communication with the inner lumen of the conduit. In implementations, the conduit is a flexible, tubular structure formed of a biotextile. In implementations, the conduit is a vascular graft. In implementations, the conduit provides an access port for a procedure in an innominate ostia, aorta, aortic root, carotid artery, or a cerebral vessel.

In another, interrelated aspect, a method for treating a patient is provided. The method includes attaching a conduit to a wall of a vessel, the conduit having an inner lumen extending between a proximal end and a distal end, the distal end of the conduit attached to the wall; forming an arteriotomy in the wall of the vessel; inserting a device through the inner lumen of the conduit and into the vessel; and performing a treatment with the device.

In another, interrelated aspect, a method for treating a patient having an atypical anatomy is provided. The method includes attaching a conduit to a wall of a vessel, the conduit having an inner lumen extending between a proximal end and a distal end, the distal end of the conduit attached to the wall; forming an arteriotomy in the wall of the vessel; inserting a device through the inner lumen of the conduit and into the vessel; and performing a treatment with the device.

In variations, one or more of the following features may be included in any feasible combination. For example, in implementations, the proximal end of the conduit includes a coupler, wherein the device is inserted through the coupler into the inner lumen of the conduit. In implementations, attaching the conduit to the wall further includes suturing the conduit to the wall with sutures. In implementations, the method further includes tightening the sutures for primary closure of the vessel following performing the treatment. In implementations, the distal end of the conduit includes a mechanical element to facilitate attaching the conduit to the vessel. In implementations, the mechanical element includes a hood, gasket, or suture ring. In implementations, forming the arteriotomy includes forming the arteriotomy through the inner lumen of the conduit attached to the wall of the vessel. In implementations, the method further includes reversing blood flow through the vessel while performing the treatment. In implementations, the vessel includes a common carotid artery. In implementations, the vessel includes a vein, left ventricular apex, axillary artery, or aorta. In implementations, the method further includes advancing the device through the common carotid artery to an innominate artery, aortic arch, descending aorta, ascending aorta, aortic root, coronary artery, internal carotid artery, external carotid artery, or an intracranial vessel. In implementations, the method further includes advancing the device through the vein, left ventricular apex, axillary artery, or aorta to an innominate artery, aortic arch, descending aorta, ascending aorta, aortic root, coronary artery, internal carotid artery, external carotid artery, or an intracranial vessel. In implementations, the device includes a balloon catheter, a stent delivery catheter, or an aspiration catheter. In implementations, the device includes vessel loops. In implementations, the treatment includes one or more of delivery of a stent, angioplasty dilation, delivery of a stent graft, delivery of a valve, aspiration embolectomy and combinations thereof. In implementations, the diameter of the inner lumen of the conduit is between about 6 mm and about 16 mm. In implementations, the diameter of the inner lumen of the conduit is between about 8 mm and about 10 mm. In implementations, the conduit further includes a vessel loop controller located at a proximal end of the conduit opposite the vessel, wherein the vessel loop controller is configured to actuate one or more vessel loops.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

These and other aspects will now be described in detail with reference to the following drawings. Generally speaking the figures are not to scale in absolute terms or comparatively, but are intended to be illustrative. Also, relative placement of features and elements may be modified for the purpose of illustrative clarity.

FIG. 1A is a schematic illustration of a retrograde blood flow system including a flow control assembly wherein an arterial access device accesses the common carotid artery via a transcarotid approach and a venous return device communicates with the internal jugular vein.

FIG. 1B is a schematic illustration of a retrograde blood flow system wherein an arterial access device accesses the common carotid artery via a transcarotid approach and a venous return device communicates with the femoral vein.

FIG. 1C is a schematic illustration of a retrograde blood flow system wherein an arterial access device accesses the common carotid artery via a transfemoral approach and a venous return device communicates with the femoral vein.

FIG. 1D is a schematic illustration of a retrograde blood flow system wherein retrograde flow is collected in an external receptacle.

FIG. 2A is an enlarged view of the carotid artery wherein the carotid artery is occluded with an occlusion element on the sheath and connected to a reverse flow shunt, and an interventional device, such as a stent delivery system or other working catheter, is introduced into the carotid artery via an arterial access device.

FIG. 2B is an alternate system wherein the carotid artery is occluded with a separate external occlusion device and connected to a reverse flow shunt, and an interventional device, such as a stent delivery system or other working catheter, is introduced into the carotid artery via an arterial access device.

FIG. 2C is an alternate system wherein the carotid artery is connected to a reverse flow shunt and an interventional device, such as a stent delivery system or other working catheter, is introduced into the carotid artery via an arterial access device, and the carotid artery is occluded with a separate occlusion device.

FIG. 2D is an alternate system wherein the carotid artery is occluded and the artery is connected to a reverse flow shunt via an arterial access device and the interventional device, such as a stent delivery system, is introduced into the carotid artery via a separate arterial introducer device.

FIG. 2E is a system where the arterial access device inserts through a conduit attached to the common carotid artery.

FIG. 3 illustrates a normal cerebral circulation diagram including the Circle of Willis.

FIG. 4 illustrates the vasculature in a patient's neck, including the common carotid artery CCA, the internal carotid artery ICA, the external carotid artery ECA, and the internal jugular vein IJV.

FIG. 5A illustrates an implementation of an arterial access device useful in the methods and systems of the present disclosure.

FIG. 5B illustrates an implementation of an arterial access device construction with a reduced diameter distal end.

FIG. 5C illustrates an implementation of a conduit for use with a retrograde blood flow system.

FIGS. 5D-5E illustrate an implementation of an arterial access device for use with a conduit.

FIG. 5F illustrates an implementation of a conduit attached to an arterial access device having no sheath.

FIG. 6 illustrates an implementation of a sheath inserted within a vessel.

FIG. 7 shows a sheath positioned within a deep vessel.

FIG. 8A shows a sheath extending through a conduit attached to a vessel.

FIG. 8B shows a sheath extending partially through a conduit attached to a vessel.

FIG. 8C shows a sheath coupled with a hemostatic valve of a conduit attached to a vessel.

FIGS. 9A-9D illustrate embodiments of a venous return device useful in the methods and systems of the present disclosure.

FIGS. 10A-10E illustrate a procedure for implanting a stent at the carotid bifurcation in accordance with the principles of the present disclosure.

FIG. 11 shows an implementation of a large bore delivery system having a large bore delivery system useful in the methods and systems of the present disclosure.

FIGS. 12A-12E illustrate the assembly and parts of the system shown in FIG. 11.

FIGS. 13A-13B illustrate an interrelated implementation of a large bore delivery system for delivery of devices useful in the methods and systems of the present disclosure.

FIGS. 14A-14B illustrate a conduit with a side port consistent with implementations of the present disclosure.

FIGS. 15A-15B illustrate a vessel loop assembly consistent with implementations of the present disclosure.

FIG. 16 illustrates an embodiment of a coupler consistent with implementations of the present disclosure.

FIGS. 17A-17C illustrate an interrelated implementation of a conduit assembly for delivery of devices useful in the methods and systems of the present disclosure.

### DETAILED DESCRIPTION

The disclosed methods, apparatus, and systems establish and facilitate access to a vessel. The methods, apparatus, and systems can provide temporary access to vessels for a variety of indications, including in the context of retrograde or reverse flow blood circulation in the region of the carotid artery bifurcation in order to limit or prevent the release of emboli into the cerebral vasculature or into peripheral vasculature that leads into the cerebral vasculature, such as the internal carotid artery. The methods are particularly useful for interventional procedures, such as stenting, delivery of stent grafts or valves, and angioplasty, atherectomy, performed through a transcarotid approach into the common carotid artery. The interventional procedures can vary including accessing and treating the cerebral vasculature such as for the treatment of stroke, Intracranial Atherosclerotic Disease (ICAD), transient ischemic attack (TIA), acute ischemic stroke (AIS), tandem lesions, ruptured and unruptured intra- and extra-cranial aneurysm embolization, chronic occlusions, and other disease conditions of the neurovasculature. The present disclosure also relates to methods and systems that are not directed into the neurovasculature, including the treatment of innominate ostial lesions, delivery of stent grafts in the aorta (TEVAR, delivery of valves to the aortic root (TAVR), and others. The present disclosure also relate to methods and systems for accessing and treating other vessels by facilitating the delivery of devices and decreasing the risk of dissection, particularly where the access vessel is particularly deep or has a short run before bifurcating. The disclosed methods, apparatus, and systems can also be useful for other approaches into the target vessel including transfemoral, trans-brachial, trans-ulnar, trans-subclavian, and trans-radial. The systems described can be useful for a variety of indications with or without reverse flow including delivery of a stent, angioplasty dilation, delivery of a stent grant, delivery of a valve, aspiration embolectomy, and combinations thereof. The systems described provide large bore access with an exceptionally flexible conduit that is temporarily affixed to the vascular access site in a manner that permits a variety of angles and curvatures to be achieved without issues of withdrawal from the access site, kinking, or collapse of the inner lumen. The conduit can have a large inner dimension so that once directly affixed to the vessel wall any of a variety of interventional tools can be inserted through it for performing any of a variety of procedures after which the conduit can be removed and the vascular access site closed. In some implementations, which will be described in more detail below, an access sheath can be positioned through the conduit and interventional tools inserted through the sheath. In other implementations, no access sheath is used such that the interventional tools are inserted through the conduit itself thereby taking advantage of the improved fluid dynamics of the large bore conduit attached to the vessel.

Access into arteries such as the common carotid artery can be established by placing a sheath or other tubular access cannula into a lumen of the artery. In the case of the common carotid artery, a distal end of the sheath typically is positioned proximal to the junction or bifurcation B from the common carotid artery to the internal and external carotid arteries. In other indications such as for neuro access, the sheath may be positioned deeper and advanced past the bifurcation B such as within an intracranial portion of the internal carotid artery (ICA) beyond the petrous ridge, for example, into the distal petrous or cavernous portion of the internal carotid through the carotid siphon into the cerebral portion of the internal carotid artery, and more distal site such as the anterior cerebrals and middle cerebral arteries.

For reverse flow systems, the sheath may have an occlusion member at the distal end, for example a compliant occlusion balloon. A catheter or guidewire with an occlusion member, such as a balloon, may be placed through the access sheath and positioned in the proximal external carotid artery ECA to inhibit the entry of emboli, but occlusion of the external carotid artery is usually not necessary. A second return sheath can be placed in the venous system, for example the internal jugular vein IJV or femoral vein FV. The arterial access and venous return sheaths can be connected to create an external arterial-venous shunt. Retrograde flow can be established and modulated to meet the patient's requirements. Flow through the common carotid artery can be occluded, either with an external vessel loop or tape, a vascular clamp, an internal occlusion member such as a balloon, or other type of occlusion means. When flow through the common carotid artery is blocked, the natural pressure gradient between the internal carotid artery and the venous system will cause blood to flow in a retrograde or reverse direction from the cerebral vasculature through the internal carotid artery and through the shunt into the venous system. Alternately, the venous sheath can be eliminated and the arterial sheath can be connected to an external collection reservoir or receptacle. The reverse flow can be collected in this receptacle. If desired, the collected blood can be filtered and subsequently returned to the patient during or at the end of the procedure. The pressure of the receptacle can be open to atmospheric pressure, causing the pressure gradient to create blood to flow in a reverse direction from the cerebral vasculature to the receptacle or the pressure of the receptacle could be a negative pressure. Optionally, to achieve or enhance reverse flow from the internal carotid artery, flow from the external carotid artery may be blocked, typically by deploying a balloon or other occlusion element in the external carotid just above (i.e., distal) the bifurcation within the internal carotid artery.

The conduit described herein can facilitate direct, temporary access to the CCA for a sheath and interventional tools or for interventional tools alone without the presence of a sheath. Where the access site vessel is the CCA and is very short between the access site and the bifurcation, no instruments need be inserted into the vessel. Rather, the conduit can be sewn onto the vessel and instruments inserted only within the conduit thereby decreasing the risk of dissection of the access site vessel. The conduit sewn onto the vessel can also facilitate direct access where the vessel is very deeply located. Deep vessels tend to increase the risk of dissection because the introducer instruments need to undergo severe turns in order to insert within the lumen of the deep vessel or otherwise impinge upon the posterior wall. The conduit sewn onto a deeply positioned vessel can decrease the risk of dissection of the posterior wall because the conduit allows access to the vessel without instrumentation needing to undergo the sharp turns.

The systems, tools, procedures and protocols described hereinafter may be useful for carotid artery disease treatments such as carotid stenting, angioplasty, atherectomy, and any other interventional procedures which might be carried out in the carotid arterial system, particularly at a location near the bifurcation between the internal and external carotid arteries. In addition, the systems and methods can be used in other vascular interventional procedures, for example the treatment of acute stroke in more distal neuro anatomies. The systems and methods described herein can provide conduit access for TCAR and neurovascular procedures for example where the CCA is very short or very deep. The systems and methods described herein can provide an access port for procedures other than TCAR or neurovascular procedures such as aspiration embolectomy. The conduit can be attached to the CCA to facilitate delivery of devices to be directed into the proximal CCA, in the aorta, and other locations. The systems and methods described herein can be used in the treatment of innominate ostial lesions, delivery of stent grafts in the aorta (TEVAR), delivery of valves to the aortic root (TAVR), and other procedures. The right and left carotid arteries can be used as points of access, the right CCA to access the innominate artery, the left CCA feeding directly to the aorta, including the aortic arch, ascending aorta, and descending aorta, the aortic root, coronary arteries, internal carotid artery, external carotid artery, an intracranial vessel, and the like. Devices used to treat innominate, aortic, or aortic valve disease tend to be very large (e.g., 20-24F or larger). Delivering these devices through and manipulating these devices within the CCA can result in vessel damage and cause bleeding in and around the device. Delivering large bore devices through the conduit sewn directly to the CCA can provide a safer, easier method to gain access to the proximal CCA, innominate arteries, aorta, and other locations.

The systems and methods may be part of a retrograde flow shunt circuit. The reverse flow system can be attached directly to the conduit so that a larger arterial sheath can be excluded from the system. The exclusion of the arterial sheath can significantly decrease the overall flow resistance of the system providing for increased reverse flow and improved embolic protection.

This application is related to U.S. Pat. No. 8,157,760 entitled “Methods and Systems for Establishing Retrograde Carotid Arterial Flow” and U.S. Patent Publication No. 2014/0296769 entitled “Methods and Systems For Establishing Retrograde Carotid Arterial Blood Flow”, both of which are incorporated herein by reference.

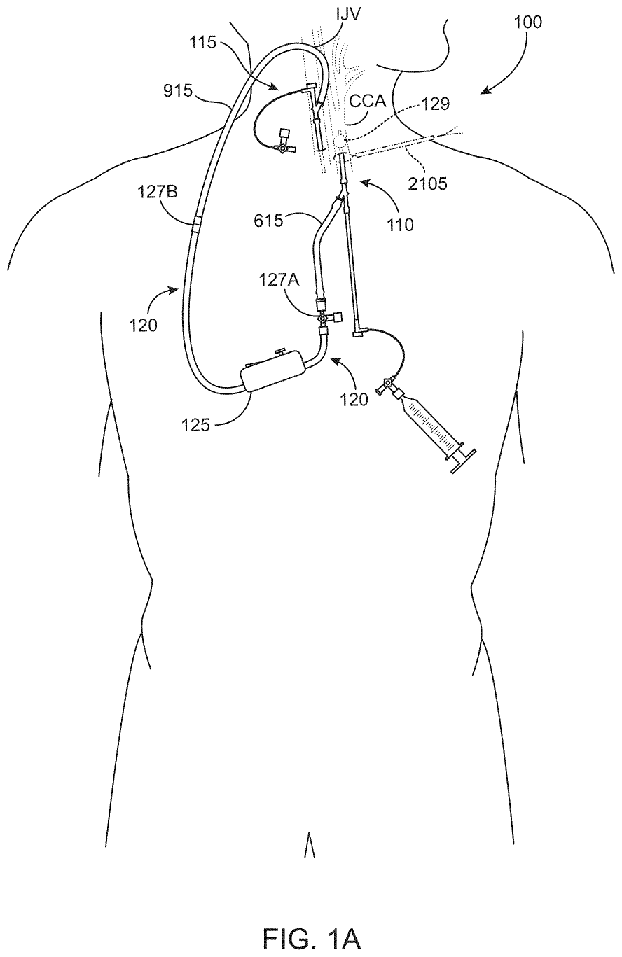

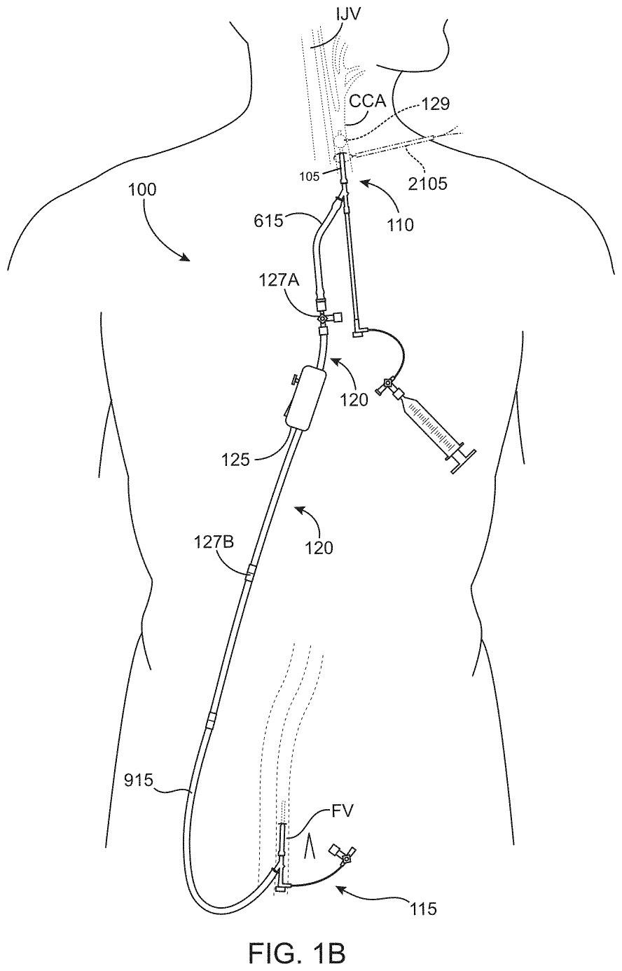

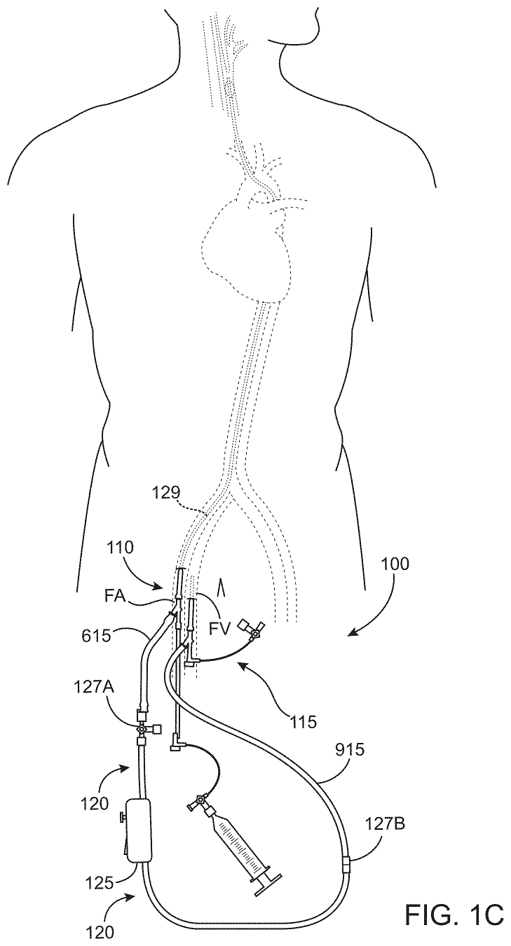

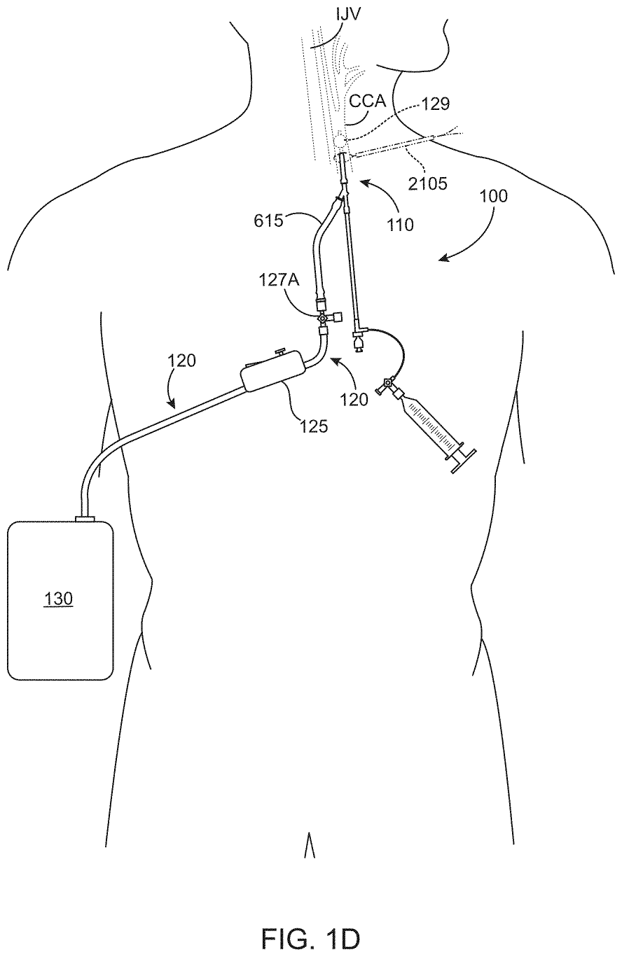

FIG. 1A shows a first embodiment of a retrograde flow system 100 that is adapted to establish and facilitate retrograde or reverse flow blood circulation in the region of the carotid artery bifurcation in order to limit or prevent the release of emboli into the cerebral vasculature, particularly into the internal carotid artery. The system 100 interacts with the carotid artery to provide retrograde flow from the carotid artery to a venous return site, such as the internal jugular vein (or to another return site such as another large vein or an external receptacle in alternate embodiments.) The retrograde flow system 100 includes a conduit 105, an arterial access device 110, a venous return device 115, and a shunt 120 that provides a passageway for retrograde flow from the arterial access device 110 to the venous return device 115. The arterial access device 110 can insert into the arterial lumen directly or via the conduit 105 attached to the arterial wall. A flow control assembly 125 interacts with the shunt 120. The flow control assembly 125 is adapted to regulate and/or monitor the retrograde flow from the common carotid artery to the internal jugular vein, as described in more detail below. The flow control assembly 125 interacts with the flow pathway through the shunt 120, either external to the flow path, inside the flow path, or both. The arterial access device 110 at least partially inserts into the common carotid artery CCA and the venous return device 115 at least partially inserts into a venous return site such as the internal jugular vein IJV, as described in more detail below. In an implementation, the arterial access device 110 can insert through the conduit 105 attached to the common carotid artery such that a distal tip of the arterial access device 110 enters the vessel lumen or the arterial access device 110 can insert only partially through the conduit 105 such that distal tip of the arterial access device 110 does not enter the vessel lumen directly. In some implementations, the arterial access device 110 and the conduit 105 couple together to place the two in fluid communication, but without a portion of the arterial access device 110 inserting substantially inside the lumen of the conduit 105. In still further implementations, the system need not incorporate an arterial access device 110. The conduit 105 attached directly to the access site vessel can form part of the retrograde flow shunt circuit. Attaching the retrograde flow shunt circuit directly to the conduit 105, without the smaller diameter arterial access device 110, the flow dynamics of the reverse flow system is improved for improved embolic protection. Where the system is described as having an arterial access device 110 and the conduit 105 interfacing with the device 110, the system can also be used without the device 110 and rely instead on the conduit 105, which will be described in more detail below

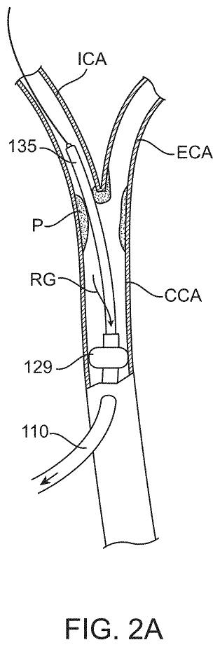

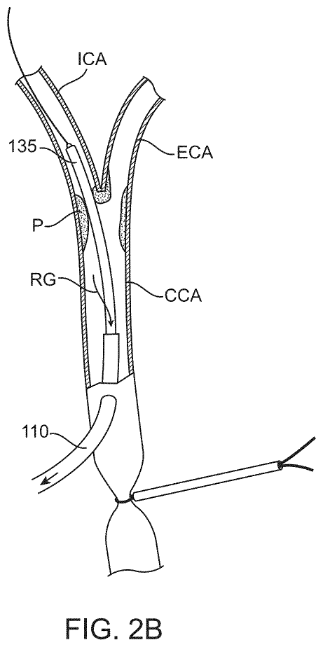

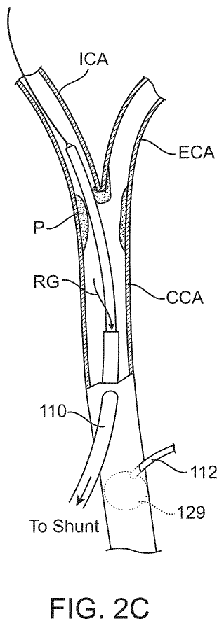

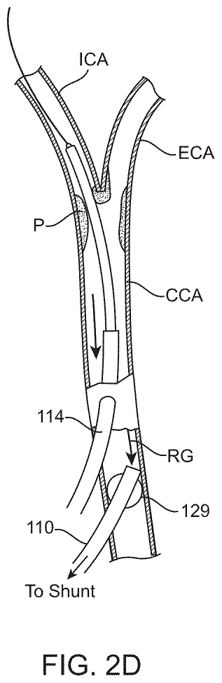

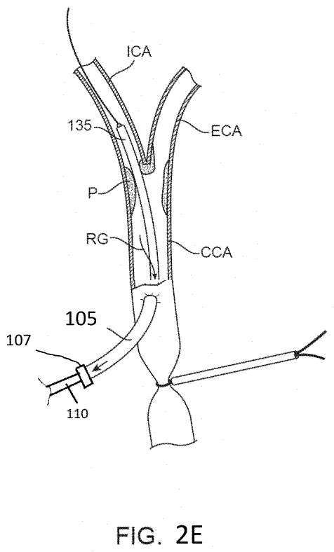

The arterial access device 110 and the venous return device 115 couple to the shunt 120 at connection locations 127a and 127b. When flow through the common carotid artery is blocked, the natural pressure gradient between the internal carotid artery and the venous system causes blood to flow in a retrograde or reverse direction RG (FIG. 2A) from the cerebral vasculature through the internal carotid artery and through the shunt 120 into the venous system. The flow control assembly 125 modulates, augments, assists, monitors, and/or otherwise regulates the retrograde blood flow.

In the embodiment of FIG. 1A, the arterial access device 110 accesses the common carotid artery CCA via a transcarotid approach. Transcarotid access provides a short length and non-tortuous pathway from the vascular access point to the target treatment site thereby easing the time and difficulty of the procedure, compared for example to a transfemoral approach. The arterial distance from the arteriotomy to the target treatment site (as measured traveling through the artery) is typically 15 cm or less, but can be as short as between 5 and 10 cm. Additionally, this access route reduces the risk of emboli generation compared with navigation of diseased, angulated, or tortuous aortic arch or common carotid artery anatomy when approached from transfemoral sites. At least a portion of the venous return device 115 is placed in the internal jugular vein IJV. In an embodiment, transcarotid access to the common carotid artery is achieved percutaneously via an incision or puncture in the skin through which the arterial access device 110 is inserted. If an incision is used, then the incision can be about 0.5 cm in length. An occlusion element 129, such as an expandable balloon, can be used to occlude the common carotid artery CCA at a location proximal of the distal end of the arterial access device 110. The occlusion element 129 can be located on the arterial access device 110 or it can be located on a separate device. In an alternate embodiment, the arterial access device 110 accesses the common carotid artery CCA via a direct surgical transcarotid approach. In the surgical approach, the common carotid artery can be occluded using a tourniquet 2105. The tourniquet 2105 is shown in phantom to indicate that it is a device that is used in the optional surgical approach. The tourniquet 2105 can be a Rummel device or arterial loop or other hemostatic technique or tool.

In another embodiment, shown in FIG. 1B, the arterial access device 110 accesses the common carotid artery CCA via a transcarotid approach while the venous return device 115 access a venous return site other than the jugular vein, such as a venous return site including the femoral vein FV. The venous return device 115 can be inserted into a central vein such as the femoral vein FV via a percutaneous puncture in the groin.

In another embodiment, shown in FIG. 1C, the arterial access device 110 accesses the common carotid artery via a femoral approach. According to the femoral approach, the arterial access device 110 approaches the CCA via a percutaneous puncture into the femoral artery FA, such as in the groin, and up the aortic arch AA into the target common carotid artery CCA. The venous return device 115 can communicate with the jugular vein JV or the femoral vein FV.

FIG. 1D shows yet another embodiment, wherein the system provides retrograde flow from the carotid artery to an external receptacle 130 rather than to a venous return site. The arterial access device 110 connects to the receptacle 130 via the shunt 120, which communicates with the flow control assembly 125. The retrograde flow of blood is collected in the receptacle 130. If desired, the blood could be filtered and subsequently returned to the patient. The pressure of the receptacle 130 could be set at zero pressure (atmospheric pressure) or even lower, causing the blood to flow in a reverse direction from the cerebral vasculature to the receptacle 130. Optionally, to achieve or enhance reverse flow from the internal carotid artery, flow from the external carotid artery can be blocked, typically by deploying a balloon or other occlusion element in the external carotid artery just above the bifurcation with the internal carotid artery. FIG. 1D shows the arterial access device 110 arranged in a transcarotid approach with the CCA although it should be appreciated that the use of the external receptacle 130 can also be used with the arterial access device 110 in a transfemoral approach.

With reference to the enlarged view of the carotid artery in FIG. 2A, an interventional device, such as a stent delivery system 135 or other working catheter, can be introduced into the carotid artery via the arterial access device 110, as described in detail below. The stent delivery system 135 can be used to treat the plaque P such as to deploy a stent into the carotid artery. The arrow RG in FIG. 2A represents the direction of retrograde flow. Alternatively, a conduit 105 can be attached directly to the CCA and the stent delivery system 135 inserted directly through the conduit 105 without an intervening arterial access device 110. The conduit 105 can be part of the retrograde flow shunt circuit providing improved embolic protection due to the larger inner diameter. The conduit 105 can be sized to receive therethrough devices that are 4F to 6F, 10F to 15F, 20F-24F or larger up to about 28F and anywhere in between. Devices used in transcarotid arterial revascularization can be delivered through the conduit 105 (e.g., a stent delivery system, balloon) and tend to be in the 4F to 6F size range. The conduit 105 can allow for passage of devices in the 10F to 15F range as well while still maintaining retrograde flow. In some implementations in which devices are introduced in the retrograde direction (e.g., toward the innominate artery or aorta), the devices introduced through the conduit 105 can be in the 24F to 36F size range.

FIG. 2B shows another embodiment, wherein the arterial access device 110 is used for the purpose of creating an arterial-to-venous shunt as well as introduction of at least one interventional device into the carotid artery. A separate arterial occlusion device 112 with an occlusion element 129 can be used to occlude the common carotid artery CCA at a location proximal to the distal end of the arterial access device 110 (see FIG. 2C).

FIG. 2D shows yet another embodiment wherein the arterial access device 110 is used for the purpose of creating an arterial-to-venous shunt as well as arterial occlusion using an occlusion element 129. A separate arterial introducer device can be used for the introduction of at least one interventional device into the carotid artery at a location distal to the arterial access device 110.

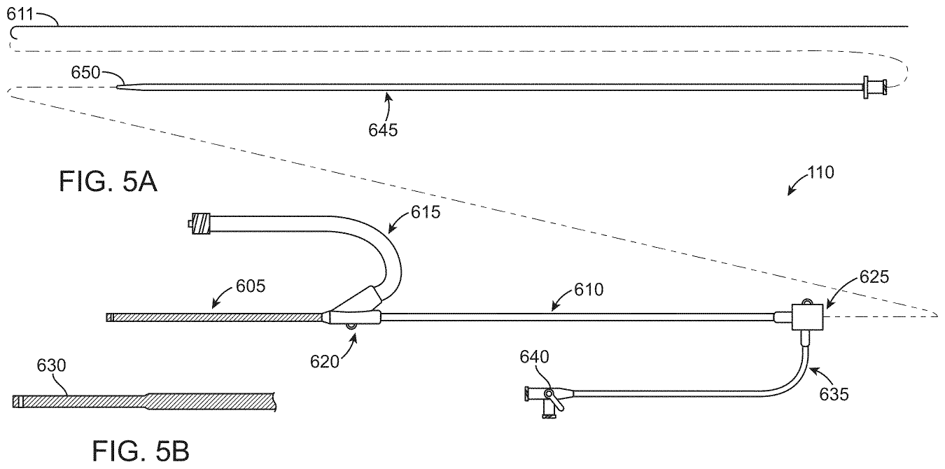

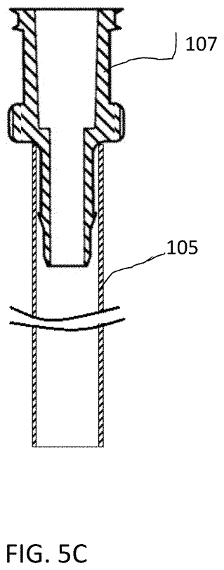

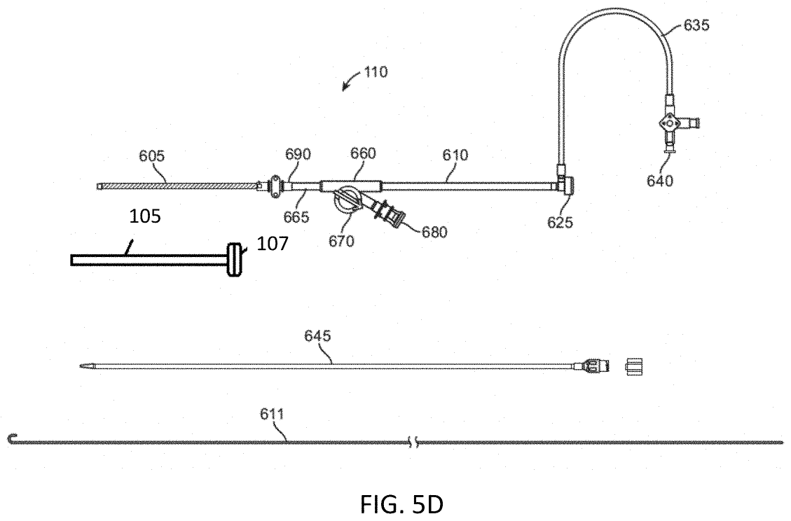

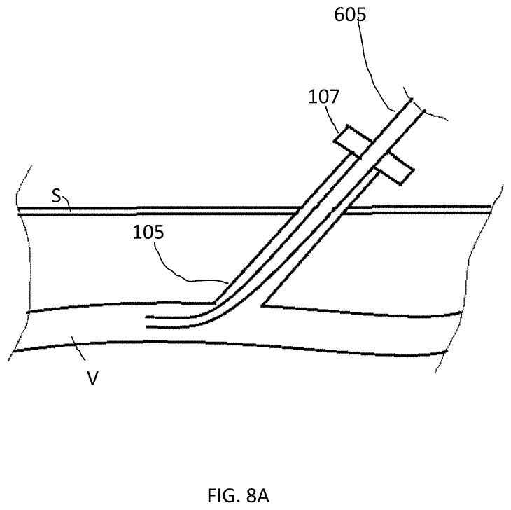

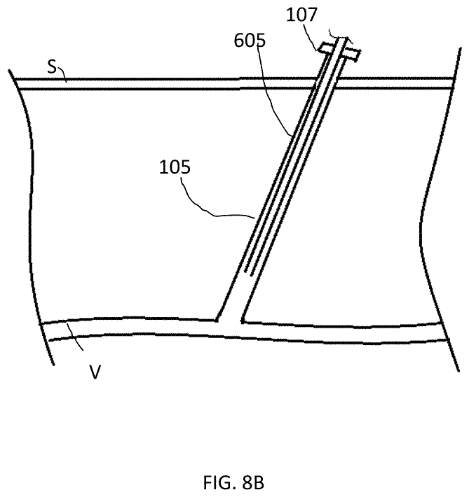

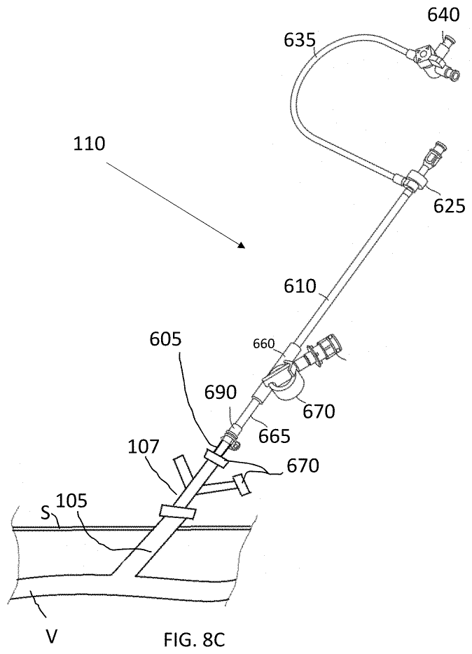

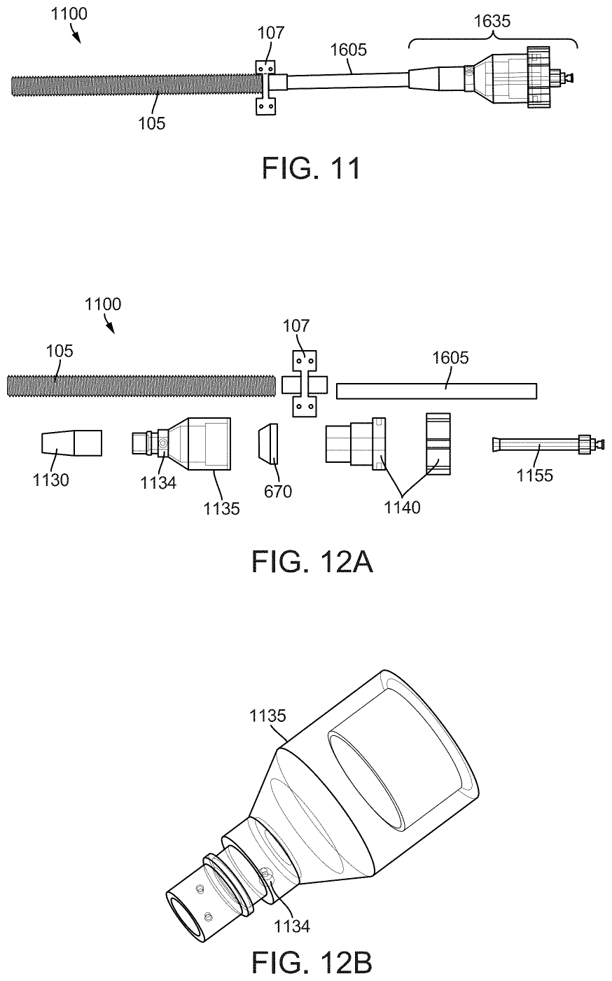

FIG. 2E shows an embodiment of the system where the arterial access device 110 inserts through the conduit 105 attached to the common carotid artery and having a coupler 107 such as a female luer fitting, a single-, dual-, or multi-headed rotating hemostatic valve (RHV), or other adaptor or coupling. The coupler 107 can be any of a variety of adaptors for vascular grafts that allow for sealing and access into the inner lumen of the conduit 105. Additional implementations of the coupler 107 and conduit 105 are shown in FIGS. 11-17C. The distal end of the conduit 105 can be attached temporarily to the vessel wall and the proximal end of the conduit 105 can extend extracorporeally so that a user may access the inner lumen of the conduit 105 through the coupler 107 for the insertion of interventional devices (e.g., catheters such as balloon catheters, large-bore aspiration catheters, and stent delivery catheters, etc.). The arterial access device 110 can extend completely through the conduit 105 so that a distal end of the device 110 inserts into the artery (see also FIGS. 5E and 8A). The arterial access device 110 can extend partially through the conduit 105 such that the distal end of the arterial access device 110 does not enter the artery directly (see also FIG. 8B). And still further embodiments are considered in which the arterial access device 110 does not insert into the conduit 105 at all and instead merely couples at the coupler 107 to place the conduit 105 and the arterial access device 110 into fluid communication with one another (see FIG. 5F). Although FIG. 2E shows the conduit 105 attached to the common carotid artery, the conduit 105 can be attached to another access site including radial, ulnar, subclavian, brachial, or femoral access site. The conduit 105 can be used in conjunction with a reverse flow system so that the larger ID of the conduit 105 can provide advantages from a fluid dynamic standpoint for improving aspiration and/or reverse flow. As mentioned above, the conduit 105 can be attached directly to the CCA and form part of the retrograde flow shunt circuit without any arterial access device 110. Interventional devices can be inserted through the conduit 105 into the vessel without needing to traverse a smaller diameter access sheath. The interventional devices can have a dimension that is significantly smaller than the conduit 105 such that the reverse flow through the circuit is improved compared to if the interventional device were inserted through an 8F arterial sheath. The large bore conduit 105 can also allow for insertion of larger devices that would otherwise be difficult to advance through a conventional sheath system. Because the neuroprotection system can be attached directly to the conduit 105 without any need for an arterial sheath, the overall flow resistance of the system can be significantly decreased and embolic protective via reverse flow improved. This is a significant advantage during delivery of balloon or stent systems that when positioned inside the arterial sheath block a large percentage of the sheath cross-sectional area. The risk of generating emboli increases during parts of procedures like the TCAR procedure when catheters and instruments are delivered into the vasculature. Improving retrograde flow rate during these “high risk” stages of the procedure can improve overall procedural outcomes. Reducing flow resistance may also reduce or eliminate the need to increase a patient's systemic blood pressure during a TCAR procedure. Typically, TCAR procedures may including elevating a patient's blood pressure to a hypertensive state (e.g., 140-160 mmHg) during high risk stages of a procedure. The elevated blood pressure increases the arterio-venous pressure differential and thereby increases reverse flow through the system to theoretically improve embolic protection. Low resistance within the large bore conduit 105 can achieve good embolic protection without the need to medially raise a patient's blood pressure thereby simplifying the procedure and benefiting the patient.

Each embodiment mentioned above and others will be discussed in more detail below.

### Description of Anatomy

Collateral Brain Circulation

The Circle of Willis CW is the main arterial anastomatic trunk of the brain where all major arteries which supply the brain, namely the two internal carotid arteries (ICAs) and the vertebral basilar system, connect. The blood is carried from the Circle of Willis by the anterior, middle and posterior cerebral arteries to the brain. This communication between arteries makes collateral circulation through the brain possible. Blood flow through alternate routes is made possible thereby providing a safety mechanism in case of blockage to one or more vessels providing blood to the brain. The brain can continue receiving adequate blood supply in most instances even when there is a blockage somewhere in the arterial system (e.g., when the ICA is ligated as described herein). Flow through the Circle of Willis ensures adequate cerebral blood flow by numerous pathways that redistribute blood to the deprived side.

The collateral potential of the Circle of Willis is believed to be dependent on the presence and size of its component vessels. It should be appreciated that considerable anatomic variation between individuals can exist in these vessels and that many of the involved vessels may be diseased. For example, some people lack one of the communicating arteries. If a blockage develops in such people, collateral circulation is compromised resulting in an ischemic event and potentially brain damage. In addition, an autoregulatory response to decreased perfusion pressure can include enlargement of the collateral arteries, such as the communicating arteries, in the Circle of Willis. An adjustment time is occasionally required for this compensation mechanism before collateral circulation can reach a level that supports normal function. This autoregulatory response can occur over the space of 15 to 30 seconds and can only compensate within a certain range of pressure and flow drop. Thus, it is possible for a transient ischemic attack to occur during the adjustment period. Very high retrograde flow rate for an extended period of time can lead to conditions where the patient's brain is not getting enough blood flow, leading to patient intolerance as exhibited by neurologic symptoms or in some cases a transient ischemic attack.

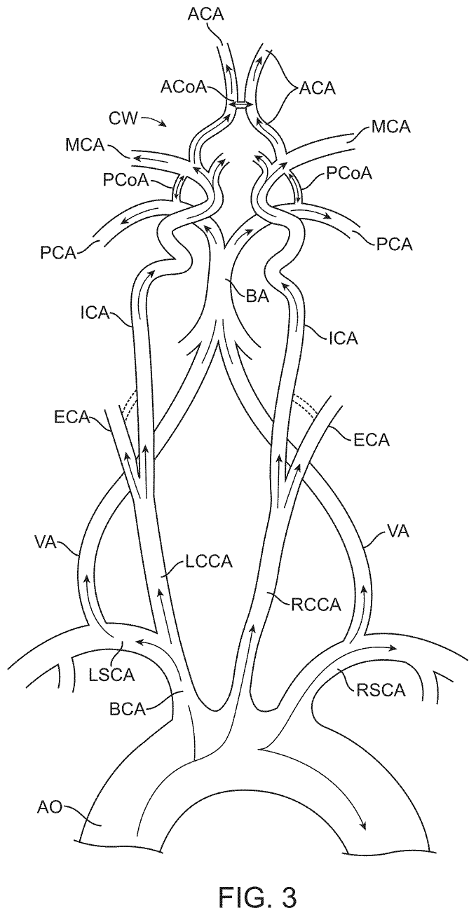

FIG. 3 depicts a normal cerebral circulation and formation of Circle of Willis CW. The aorta AO gives rise to the brachiocephalic artery BCA, which branches into the left common carotid artery LCCA and left subclavian artery LSCA. The aorta AO further gives rise to the right common carotid artery RCCA and right subclavian artery RSCA. The vertebral arteries VA branch from the LSCA and the RSCA. The left and right common carotid arteries CCA gives rise to internal carotid arteries ICA which branch into the middle cerebral arteries MCA, posterior communicating artery PcoA, and anterior cerebral artery ACA. The anterior cerebral arteries ACA deliver blood to some parts of the frontal lobe and the corpus striatum. The middle cerebral arteries MCA are large arteries that have tree-like branches that bring blood to the entire lateral aspect of each hemisphere of the brain. The left and right posterior cerebral arteries PCA arise from the basilar artery BA and deliver blood to the posterior portion of the brain (the occipital lobe).

Anteriorly, the Circle of Willis is formed by the anterior cerebral arteries ACA and the anterior communicating artery ACoA which connects the two ACAs. The two posterior communicating arteries PCoA connect the Circle of Willis to the two posterior cerebral arteries PCA, which branch from the basilar artery BA and complete the Circle posteriorly.

The common carotid artery CCA also gives rise to external carotid artery ECA, which branches extensively to supply most of the structures of the head except the brain and the contents of the orbit. The ECA also helps supply structures in the neck and face.

Carotid Artery Bifurcation

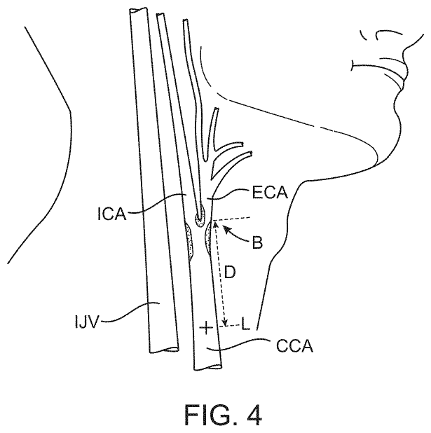

FIG. 4 shows an enlarged view of the relevant vasculature in the patient's neck. The common carotid artery CCA branches at bifurcation B into the internal carotid artery ICA and the external carotid artery ECA. The bifurcation is located at approximately the level of the fourth cervical vertebra. FIG. 4 shows plaque P formed at the bifurcation B.

As discussed above, the common carotid artery CCA can be accessed via a transcarotid approach. Pursuant to the transcarotid approach, the common carotid artery CCA can be accessed at an arterial access location L, which can be, for example, a surgical incision or puncture in the wall of the common carotid artery CCA. There is typically a distance D of around 5 to 7 cm between the arterial access location L and the bifurcation B. When the arterial access device 110 is inserted into the common carotid artery CCA, it is undesirable for the distal tip of the arterial access device 110 to contact the bifurcation B as this could disrupt the plaque P and cause generation of embolic particles. This can be particularly risky for shorter CCA. In order to minimize the likelihood of the arterial access device 110 contacting the bifurcation B, in an embodiment only about 2-4 cm of the distal region of the arterial access device may be inserted into the common carotid artery CCA during a procedure.

In other implementations, the arterial access device 110 is placed in fluid communication with the common carotid artery via a conduit 105 attached to the vessel wall, such as the anterior vessel wall (see FIG. 2E and FIGS. 8A-8C). The distal tip of the arterial access device 110 can insert through the lumen of the conduit 105 and a small distance into the vessel lumen (FIG. 8A) or does not enter the vessel lumen at all and remains within the lumen of the conduit 105 (FIG. 8B). The conduit 105 can provide safer insertion of the arterial access device 110 and lower the risk of plaque disruption. The conduit 105 may also lower risk of damage to the artery due to, for example, vessel dissection or vessel perforation.

In still further implementations, the conduit 105 is attached directly to the CCA and the system excludes the arterial access device 110 entirely. Interventional tools are inserted directly through the conduit 105, which forms a part of the retrograde flow shunt circuit. Where the arterial access device 110 is described as inserting through the conduit 105, it should be appreciated an interventional device or tool can be inserted through the conduit without any arterial access device 110 incorporated into the system.

The common carotid arteries are encased on each side in a layer of fascia called the carotid sheath. This sheath also envelops the internal jugular vein and the vagus nerve. Anterior to the sheath is the sternocleidomastoid muscle. Transcarotid access to the common carotid artery and internal jugular vein, either percutaneous or surgical, can be made immediately superior to the clavicle, between the two heads of the sternocleidomastoid muscle and through the carotid sheath, with care taken to avoid the vagus nerve. One complication during insertion of the arterial access device 110 is dissection of the CCA, particularly for anatomies in which the CCA is located relatively deep beneath the surface of the skin S. When the CCA is located deep beneath the skin and the sternocleidomastoid muscle, the angle of insertion can be greater than 50 degrees, such as up to about 80 degrees. It is this angle that can result in vessel wall dissection or even perforation of the posterior wall of the artery during insertion.

The conduit 105 can facilitate safe insertion of catheters (e.g., interventional tools, the arterial access device 110, etc.) into the CCA even in these challenging anatomies causing steep angle of approach. The conduit 105 can be a temporary vascular conduit attached to the anterior wall of the CCA via suture using standard surgical technique. “Temporary” as used herein refers to a period of time that is sufficient to perform the interventional procedure. A conduit 105 that is affixed to a vessel and provides temporary access for a procedure is temporary in that, following the procedure, flow from the vessel through the conduit 105 is occluded, either because the conduit 105 is removed from the vessel and the opening in the vessel closed or because the inner lumen of the conduit 105 is occluded or sealed off so that after the procedure flow through the conduit 105 is prevented. The arterial access device 110 rather than inserting through the vessel wall directly can insert through the conduit lumen and need not be advanced as far as the vessel lumen in order to be place in fluid communication with the vessel lumen. The conduit 105 mitigates the risk of CCA dissection or inadvertent liberation of embolic material from plaques at the carotid bifurcation.

Devices inserted through the conduit 105 can be advanced from the common carotid artery proximally, for example, into an innominate artery, a portion of the aorta including the aortic arch, the descending aorta, or the ascending aorta, aortic root, or into a coronary artery. The conduit 105 can also provide access for devices intended to be inserted for neurovascular procedures into the internal carotid artery, external carotid artery, or an intracranial vessel.

Detailed Description of Retrograde Blood Flow System with Additional Conduit

As discussed, an implementation of the retrograde flow system 100 includes the arterial access device 110, venous return device 115, shunt 120, which provides a passageway for retrograde flow from the arterial access device 110 to the venous return device 115, and a conduit 105 configured to attach to the arterial wall and place the arterial access device 110 in fluid communication with the arterial lumen (see FIG. 5D). The system also includes the flow control assembly 125, which interacts with the shunt 120 to regulate and/or monitor retrograde blood flow through the shunt 120. The arterial access device 110 can access the vessel directly or through the separate conduit 105. In implementations, the retrograde flow system 100 can incorporate a conduit 105 having a distal end configured to attach directly to a vascular wall and having a proximal end configured to couple to the shunt 120, such as via a connector 107. In this implementation, no arterial access device 110 is incorporated. Exemplary embodiments of the components of the retrograde flow system 100 are now described.

Conduit

FIG. 5C illustrates an implementation of a conduit 105. The conduit 105 can be designed for use with a retrograde flow system 100. The conduit 105 can be a flexible, tubular structure capable of being attached to a vessel wall. The conduit material can be compliant and allow for radial expansion. This allows for larger devices to be delivered through the conduit 105 while minimizing the size of the arteriotomy/anastomosis. The conduit can be coated on an inner and/or outer surface with anti-thrombogenic materials (e.g., heparin) and/or hydrophilic material to facilitate delivery of instruments and devices through the lumen of the conduit 105. The conduit 105 can be configured to accommodate patient anatomies and/or medical conditions that are not conducive to large bore access, such as procedures on short/deep vessels, patients with disease, dissections, patients having scar tissue, etc., and can reduce the difficulty or risk involved with such procedures or patients. FIGS. 11-17C illustrate other interrelated implementations of the conduit, which will be described in more detail below.

The conduit body can be a knit or woven fabric that can provide flexibility and that may be coated with a polymer to provide resistance to blood leaking through the fabric. For example, the knit or woven fabric can be PET and the coating can be polyurethane or similar polymer. The coating to prevent leaking can be focused to the locations through which sutures will be drawn. The coating can also mitigate against fraying of the fabric at the distal and/or proximal ends. The fabric can be Dacron polyethylene terephthalate fabric impregnated with collagen to create a leak-free conduit (e.g., Maquet, Getinge AB, Hemashield Gold graft).

The conduit 105 can be a synthetic arterial prosthesis or shaped biotextile. The conduit 105 can be arterial vascular graft produced from a base material and extruded into a tube shape. The base material can be a synthetic polymer material such as polyethylene terephthalate (Dacron), polyester, nylon, expanded polytetrafluoroethylene (ePTFE), heparin-bonded ePTFE, hooded PTFE, ring reinforced ePTFE, nylon-ePTFE woven hybrid, or other suitable material. The conduit 105 can be formed of a commercially-available vascular graft such as CARBOFLOW (Bard), FLIXENE PTFE (Atrium Medical), PROPATEN (Gore), dual-layer construction heparin vascular grafts of ePTFE and PET layers (FUSION BIOLINE, Getinge AB), HERO hemodialysis access graft (Merit Medical), or ACUSEAL (Gore). The conduit 105 can be formed of a bio-absorbable materials such as polyglycolide (PGA), polyglycolide-polylactic acid (PGA-PLA), poly(lactic-co-glycolic acid) (PGLA), polycaprolactone (PCL), or the like.

The distal end of the conduit 105 can be attached to the vessel wall according to techniques known in the art. In some implementations, the conduit 105 is sutured to the vessel wall such that the lumen of the conduit 105 is placed in fluid communication with the vessel lumen. The conduit 105 can be sutured to the vessel wall using standard surgical technique to create end-to-side anastomoses (see Criado “Iliac arterial conduits for endovascular access: technical considerations” J. Endovasc. Ther. 2007 14(3):347-351). The conduit 105 can also be attached using sutureless techniques to create end-to-side anastomoses. The conduit 105 can be grafted to the artery so that it is positioned at an angle relative to the longitudinal axis of the vessel lumen. The angle between the conduit 105 and the longitudinal axis of the vessel lumen can vary from 0 to 90 degrees, preferably 20-45 degrees. The flexibility of the conduit 105, compared to traditional access sheaths, in combination with the flexibility of the vessel, allows for the angle of attachment to be relatively severe while still allowing for safe passage of devices through the conduit 105 into the vessel.

The conduit 105 can be sewn directly onto the vessel prior to creating an arteriotomy forming an anastomosis. Once the anastomosis is completed, an arteriotomy can be created from inside the conduit. This method needs no distal or proximal claim prior to the anastomosis. The sutures used to sew the conduit 105 to the vessel to form the anastomosis can also be used for primary closure of the arteriotomy in the vessel at the end of the procedure after the conduit 105 is removed from its attachment.

The distal end region of the conduit 105 can include a mechanical feature configured to facilitate faster anastomosis with decreased bleeding. The feature can include a hood, gasket, suture ring, or similar feature positioned at a distal end region of the conduit 105. The distal end region of the conduit 105 can include pre-placed or pre-sewn closure sutures positioned such that, at the conclusion of the procedure performed through the conduit 105, the pre-placed sutures may be tightened to quickly close the conduit 105 and the vessel similar to an “over-sew” technique. Pre-placing the over-sew sutures in the distal end region of the conduit 105 can help to ensure more precise placement of closure sutures for a more complete and more efficient closure of the conduit 105. In the over-sew technique, a small remnant of conduit material may be left behind following closure although no blood flows through the remnant.

The conduit 105 can incorporate reinforcing feature on a proximal end region. The reinforcing features on the conduit can include one or more coils, braids, rings, or combination thereof that are configured to mitigate against kinking or collapse if the conduit 105 is bent. The reinforcing features can extend from the proximal end region terminating 1-2 cm proximal to the distal-most end of the conduit 105. The distal 1-2 cm of the conduit 105 therefore can be un-reinforced to maintain compliance and flexibility of the conduit material. This un-reinforced region can be sewn to the vessel.

The conduit 105 can have an inner diameter sufficient to receive an outer diameter of an arterial access device or sufficient to receive an outer diameter of any of a variety of interventional devices and tools used for procedures such as transcarotid arterial revascularization (TCAR), the treatment of innominate ostial lesions, delivery of stent grafts in the aorta (TEVAR), delivery of valves to the aortic root (TAVR), neurovascular procedures, and the like. In some implementations, the conduit 105 can have an inner diameter between about 6 mm and about 16 mm. In some implementations, the conduit 105 can have an inner diameter between about 6 mm and about 15 mm. In some implementations, the conduit 105 can have an inner diameter between about 7 mm and about 12 mm. In some implementations, the conduit 105 can have an inner diameter between about 8 mm and about 10 mm. In some implementations, the arterial access device 110 is in the range of 7 Fr to 10 Fr sheath. In this instance, the inner diameter of the conduit 105 is sized to receive this size sheath. Generally, an 8 Fr sheath has an outer diameter that is about 10.5 F (1 Fr=0.33 mm). A sheath having this size can be inserted through the arterial wall into the lumen of the vessel without causing unnecessary damage to the artery and still large enough to permit robust retrograde flow for embolic protection. The conduit 105 can be significantly larger than this size because the conduit 105 is attached to the vessel wall rather than inserting through the vessel wall and into the vessel lumen, typically having an inner diameter of about 6 mm. Where the outer diameter of a sheath entering the vessel is limited by the inner diameter of the vessel lumen, the outer diameter of the conduit 105 is not. The outer diameter of the conduit 105 can be about 16 Fr up to about 20 Fr and can have an inner diameter that is in the range of 15 Fr to 18 Fr (5-6 mm). Devices used to treat innominate, aortic, or aortic valve disease can be in a range of between 20 Fr and 24 Fr or larger. The conduit 105 can be sized to receive up to 24 Fr or larger sizes for these procedures. In some implementations, the arterial access device 110 is in the range of 12 Fr to 36 Fr sheath. In some implementations, the arterial access device 110 is in the range of 14 Fr to 20 Fr sheath.

The conduit 105 can be any reasonable inner diameter that allows for grafting to the vessel wall and suitable fluid flow through the conduit 105 for embolic protection during a procedure. In some implementations, the inner diameter of the conduit is between about 2 mm to about 10 mm, or about 4 mm to about 8 mm. The hole in the artery can likewise be about 6-8 mm dependent on the size of the conduit. In some implementations, the inner diameter of the conduit approaches the inner diameter of the vessel it is to be attached to. It should be appreciated that the arterial access device 110 need not be inserted into the lumen of the conduit 105 to be placed in fluid communication with the vessel. For example, the arterial access device 110 has no distal sheath so that upon coupling the arterial access device to the coupler 107 the blood flows directly from the CCA into the inner diameter of the conduit 105 so that the system effectively has no arterial sheath. This reduces the resistance to fluid flow that would otherwise occur if the blood flowed into a smaller arterial sheath (e.g., 2.7 mm ID). Reducing the resistance to fluid flow increases retrograde flow through the system. The inner dimension of other components in the system can also be increased to further reduce flow resistance from the point where the blood enters the conduit proximally.

The conduit 105, while in use, can have a length between the proximal opening and the distal opening of the conduit 105 that is about 1 cm to 15 cm, preferably about 5 cm to about 10 cm, and in some applications up to about 20 cm to about 30 cm. Longer lengths greater than 20 cm are considered, for example, to couple directly to the vessel while also position a user further away from the X-ray beam during fluoroscopy. The length of the conduit 105 is sufficient to attach to an anterior wall of the CCA, even in anatomies where the CCA is deep, and to extend a length outside the incision in the neck to couple with the arterial access device 110 without resulting in external tension on the system. The length of the conduit 105 can be customized at the time of use. As such, the length of the conduit 105 can be manufactured to be longer than what might be used during a procedure, for example, at least about 30 cm or between about 20 cm to about 50 cm, and trimmed to size at the time of use, for example, between about 5 cm up to about 30 cm. In still other implementations, the conduit 105 can be provided in various pre-sized lengths ranging from about 5 cm up to about 30 cm. A length of the conduit 105 between the proximal end and the distal end can be longer than a length of the distal sheath so that when the distal sheath extends through the inner lumen of the conduit 105, a distal end of the distal sheath remains proximal to the distal end of the conduit and inside the conduit inner lumen. For example, the conduit can have an insertable length between about 5 cm and about 30 cm and the insertable length of the distal sheath can be shorter than this, for example, between about 4 cm and about 29 cm.

Devices such as the arterial sheath 110 or another device can be inserted through the proximal opening of the conduit 105 or through a sidewall of the conduit 105. The arterial sheath 110 or other device can be secured within the conduit 105 using a Rummel tourniquet, vessel loop, or similar device. Regardless how the arterial sheath 110 (or the retrograde shunt if no sheath 110 is incorporated) is placed in fluid communication with the conduit 105, leakage of blood around the sheath as well as inadvertent movements of the sheath relative to the conduit are to be controlled.

In an implementation, the conduit 105 can have a coupler 107 positioned within the proximal opening of the conduit 105. The coupler 107 can be a luer fitting, a Hemostasis Valve Adaptor (HVA), or a Rotating Hemostasis Valve (RHV), or other type of fitting. The coupler 107 can be configured for attachment to standard connectors (e.g., syringe, stopcocks, hemostasis valve, Y-connector, or other type of connector). The coupler 107 can provide both securement of a sheath such as the arterial access device 110 or a shunt providing proximal attachment to the conduit 105 and hemostasis against blood leakage during use. Additional, interrelated implementations of the coupler 107 are described elsewhere herein, including FIGS. 11, 12A, 12E-14A, and 16. The coupler 107 can be an integrated non-standard connector that limits its compatibility to specified mating components. The coupler 107 can be a multi-port adaptor each port with a rotating hemostasis valve. The multi-port adapter allows for additional devices to be introduced into the conduit 105 (and optionally into the CCA). For example, one or more of the ports can be used for the delivery of a “buddy” wire or support catheter to facilitate delivery of interventional devices to the target locations (e.g., 0.014″ guidewire, balloon catheter, stent delivery system). One or more of the ports can be used to attach a supplemental extracorporeal shunt line to augment retrograde flow through the system 100. One or more of the ports can be used to deliver an aspiration catheter into the vessel, for example into the CCA or beyond into the ICA to provide supplemental embolic protection during a procedure, or to provide aspiration for removal of clot/thrombus in cases of acute ischemic stroke. The conduit 105 can be incorporated into a retrograde flow system that may be used with any of a variety of tools and for any of a variety of procedures in the neurovasculature, the coronary arteries, as well as peripheral vessels. The inner dimension of the conduit 105 and stable coupling between the conduit 105 and the access site vessel provide additional flexibility in the procedure that may be performed while still providing sufficient embolic protection. The blood flow through the aspiration catheter may be passive or flow may be supplemented by an external vacuum pump.

The distal portion of the arterial access device 110 (e.g., distal sheath 605 shown in FIG. 5A) can, but need not insert through the conduit 105. In some implementations, the distal sheath 605 is inserted at least partially through the coupler 107 and into the lumen of the conduit 105 (see FIGS. 5E and 8A-8B). The distal sheath 605 inserted into the lumen of the conduit 105 can be inserted far enough that the coupler on the arterial access device 110 proximal to the distal sheath 605 (e.g. distal connector 690 shown in FIG. 5D) can engage with and attach to the coupler 107 of the conduit 105. The distal sheath 605 further includes a sheath lumen, and the sheath lumen diameter may be sized between about 12 Fr to about 36 Fr. In some implementations, the sheath lumen diameter may be sized between about 14 Fr to about 20 Fr. In other implementations, the coupler 107 of the conduit 105 can attach to the coupler of the arterial access device 110 directly without any sheath 605 inserting through the lumen of the conduit 105 (see FIG. 5F). The conduit 105 attached to the arterial wall can take the place of any arterial sheath that would normally be inserted into the vessel lumen. As discussed elsewhere herein, eliminating the presence of the sheath body within the flow circuit can greatly reduce the flow resistance providing for increased retrograde flow rates and improved embolic protection. The conduit 105 is temporary and is removed after use such as by removing the sutures and closing the hole in the vessel.

Arterial Access Device

FIG. 5A shows an exemplary embodiment of the arterial access device 110, which can include a distal sheath 605, a proximal extension 610, a flow line 615, an adaptor or Y-connector 620, and a hemostasis valve 625. The arterial access device may also include a dilator 645 with a tapered tip 650 and an introducer guide wire 611. The arterial access device together with the dilator and introducer guidewire can be used together to gain access to a vessel. Features of the arterial access device may be optimized for transcarotid access. For example, the design of the access device components may be optimized to limit the potential injury on the vessel due to a sharp angle of insertion, allow atraumatic and secure sheath insertion, and limiting the length of sheath, sheath dilator, and introducer guide wire inserted into the vessel.

In some implementations, the distal sheath 605 includes an occlusion element 129 for occluding flow through a vessel, for example the common carotid artery. If the occluding element 129 is an inflatable structure such as a balloon or the like, the sheath 605 can include an inflation lumen that communicates with the occlusion element 129. The occlusion element 129 can be an inflatable balloon, but it can also be an inflatable cuff, a conical or other circumferential element which flares outwardly to engage the interior wall of the common carotid artery to block antegrade flow past the occlusion element 129, a membrane-covered braid, a slotted tube that radially enlarges when axially compressed, or similar structure which can be deployed by mechanical means, or the like. In the case of balloon occlusion, the balloon can be compliant, non-compliant, elastomeric, reinforced, or have a variety of other characteristics. In an embodiment, the balloon is an elastomeric balloon which is closely received over the exterior of the distal end of the sheath prior to inflation. When inflated, the elastomeric balloon can expand and conform to the inner wall of the vessel. In an embodiment, the elastomeric balloon is able to expand to a diameter at least twice that of the non-deployed configuration, frequently being able to be deployed to a diameter at least three times that of the undeployed configuration, more preferably being at least four times that of the undeployed configuration, or larger.

FIG. 2B shows an alternative embodiment, wherein the occlusion element 129 can be introduced into the vessel on a second sheath 112 separate from the distal sheath 605 of the arterial access device 110. The second or “proximal” sheath 112 can be adapted for insertion into the vessel in a proximal or “downward” direction away from the cerebral vasculature. The second, proximal sheath can include an inflatable balloon 129 or other occlusion element, generally as described above. The distal sheath 605 of the arterial access device 110 can be then placed into the vessel distal of the second, proximal sheath and generally oriented in a distal direction toward the cerebral vasculature. By using separate occlusion and access sheaths, the size of the arteriotomy needed for introducing the access sheath can be reduced.

FIG. 2C shows yet another embodiment of a two arterial sheath system, wherein the interventional devices are introduced via an introducer sheath 114 separate from the distal sheath 605 of the arterial device 110. A second or “distal” sheath 114 can be adapted for insertion into the vessel distal to the arterial access device 110. As with the previous embodiment, the use of two separate access sheaths allows the size of each arteriotomy to be reduced.

In a situation with a sharp sheath insertion angle and/or a short length of sheath inserted in the artery, such as one might see in a transcarotid access procedure into the common carotid artery, the distal tip of the sheath has a higher likelihood of being partially or totally positioned against the vessel wall, thereby restricting flow into the sheath. In an embodiment, the sheath is configured to center the tip in the lumen of the vessel. One such embodiment includes a balloon such as the occlusion element 129 described above. In another embodiment, a balloon may not be occlusive to flow but still center the tip of the sheath away from a vessel wall, like an inflatable bumper. In another embodiment, expandable features are situated at the tip of the sheath and mechanically expanded once the sheath is in place. Examples of mechanically expandable features include braided structures or helical structures or longitudinal struts which expand radially when shortened.

The distal sheath 605 is adapted to be introduced through an incision, arteriotomy, or puncture in a wall of a vessel, either an open surgical incision or a percutaneous puncture established, for example, using the Seldinger technique. The length of the sheath 605 can be in the range from 5 to 15 cm, usually being from 10 cm to 12 cm. The sheath 605 can also be placed in fluid communication with the vessel through the conduit 105 as discussed elsewhere herein. In still further implementations, the arterial access device 110 has no distal sheath 605, which will be described in more detail below.

The inner diameter of the distal sheath 605 can be in the range from 7 Fr (1 Fr=0.33 mm), to 10 Fr, usually being 8 Fr. When the distal sheath 605 is inserted through an arteriotomy it is preferred that the OD is within a range of about no greater than about 10 Fr. As will be discussed in more detail below, the distal sheath 605 can be larger when used together with the conduit 105, for example between 15 Fr to about 18 Fr.

It is desirable that the sheath 605 be highly flexible while retaining hoop strength to resist kinking and buckling, particularly when the sheath is being introduced through the transcarotid approach into the common carotid artery, above the clavicle but below the carotid bifurcation. Thus, the distal sheath 605 can be circumferentially reinforced, such as by braid, helical ribbon, helical wire, cut tubing, or the like and have an inner liner so that the reinforcement structure is sandwiched between an outer jacket layer and the inner liner. The inner liner may be a low friction material such as PTFE. The outer jacket may be one or more of a group of materials including Pebax, thermoplastic polyurethane, or nylon. In an embodiment, the reinforcement structure or material and/or outer jacket material or thickness may change over the length of the sheath 605 to vary the flexibility along the length.

The conduits described herein can be attached to any of a variety points of access including carotid, femoral, radial, brachial, ulnar, or subclavian arteries. Certain vascular structures such as veins, the left ventricular apex, the axillary artery and the aorta may be used for alternative large bore access, e.g. access using a large bore delivery system 1100 as described in further detail elsewhere herein. These structures are not as robust as the carotid artery, and procedures on these structures may benefit from attachment of a conduit 105 which may be made of inorganic material or graft material, such as that of the large bore delivery systems described herein (see FIGS. 11-17C). The conduit can be attached to a wall of these access vessels and another access device such as the sheath 605 advanced through it. Preferably, the conduit provides access without any other access device or sheath inserted through it such that interventional devices can be inserted directly through the conduit and towards the target anatomy for treatment. The interventional device can include a balloon catheter, a stent delivery catheter, an aspiration catheter and the like. The interventional device can be inserted under reverse flow using a reverse flow system attached directly to a coupler on the conduit 105. The target vessel can vary as well including an extracranial or intracranial vessel. The target vessel can include the common carotid artery CCA, external carotid artery ECA, internal carotid artery ICA, cerebral arteries (M1 or M2 segments of the middle cerebral arteries), vertebral artery, subclavian artery, brachiocephalic artery, innominate artery, ascending aorta, aortic arch, descending aorta, or aortic root. Other target vessels include vessels of the coronary anatomy, peripheral anatomy, or other vasculature. Coronary vessels including the left and right coronary arteries, posterior descending artery, right marginal artery, left anterior descending artery, left circumflex artery, M1 and M2 left marginal arteries, and D1 and D2 diagonal branches. Any of a variety of peripheral vessels are considered herein as target vessels including the popliteal arteries, anterior tibial arteries, dorsalis pedis artery, posterior tibial arteries, and fibular artery. Where a particular anatomy is mentioned in the context of the devices described herein, other anatomies are considered although may not be specified at each instance.

In some implementations, the distal sheath 605 can have a stepped or other configuration having a distal region 630 that is reduced in outer diameter compared to more proximal regions, as shown in FIG. 5B, which shows an enlarged view of the distal region 630 of the sheath 605. The distal region 630 of the sheath can be sized for insertion into the carotid artery, typically having an inner diameter in the range from 2.16 mm (0.085 inch) to 2.92 mm (0.115 inch) with the remaining proximal region of the sheath having larger outside and luminal diameters, with the inner diameter typically being in the range from 2.794 mm (0.110 inch) to 3.43 mm (0.135 inch). The larger luminal diameter of the proximal region minimizes the overall flow resistance of the sheath. In an embodiment, the reduced-diameter distal section 630 has a length of approximately 2 cm to 4 cm. The relatively short length of the reduced-diameter distal section 630 permits this section to be positioned in the common carotid artery CCA via the transcarotid approach with reduced risk that the distal end of the sheath 605 will contact the bifurcation B. Moreover, the reduced diameter section 630 also permits a reduction in size of the arteriotomy for introducing the sheath 605 into the artery while having a minimal impact in the level of flow resistance. Further, the reduced distal diameter section may be more flexible and thus more conformal to the lumen of the vessel.

With reference again to FIG. 5A, the proximal extension 610, which is an elongated body, has an inner lumen which is contiguous with an inner lumen of the sheath 605. The lumens can be joined by the Y-connector 620 which also connects a lumen of the flow line 615 to the sheath. The flow line connection can terminate in a valve and the sheath can include a Y-adaptor that connects the distal portion of the sheath to the proximal extension 610. The Y-adapter can also include a valve that can be operated to open and close fluid connection to a connector or hub that can be removably connected to a flow line such as a shunt. The valve can be positioned immediately adjacent to an internal lumen of the adapter, which communicates with the internal lumen of the sheath body 605.

In the assembled system, the flow line 615 connects to and forms a first leg of the retrograde shunt 120 (see FIG. 1A). The proximal extension 610 can have a length sufficient to space the hemostasis valve 625 well away from the Y-connector 620, which is adjacent to the percutaneous or surgical insertion site. By spacing the hemostasis valve 625 away from a percutaneous insertion site, the physician can introduce tools such as a stent delivery system or other working catheter into the proximal extension 610 and sheath 605 while staying out of the fluoroscopic field when fluoroscopy is being performed. In an embodiment, the proximal extension 610 is about 16.9 cm from a distal most junction (such as at the hemostasis valve 625) with the sheath 605 to the proximal end of the proximal extension 610. In an embodiment, the proximal extension has an inner diameter of 0.125 inch and an outer diameter of 0.175 inch. In an embodiment, the proximal extension has a wall thickness of 0.025 inch. The inner diameter may range, for example, from 0.60 inch to 0.150 inch with a wall thickness of 0.010 inch to 0.050 inch. In another embodiment, the inner diameter may range, for example, from 0.150 inch to 0.250 inch with a wall thickness of 0.025 inch to 0.100 inch. The dimensions of the proximal extension may vary. In an embodiment, the proximal extension has a length within the range of about 12-20 cm. In another embodiment, the proximal extension has a length within the range of about 20-30 cm.

In an embodiment, the distance along the sheath from the hemostasis valve 625 to the distal tip of the sheath 605 is in the range of about 25 and 40 cm. In an embodiment, the distance is in the range of about 30 and 35 cm. With a system configuration that allows 2.5 cm of sheath introduction into the artery, and an arterial distance of between 5 and 10 cm from the arteriotomy site to the target site, this system enables a distance in the range of about 32.5 cm to 42.5 cm from the hemostasis valve 625 (the location of interventional device introduction into the access sheath) to the target site of between 32 and 43 cm. This distance is about a third the distance required in prior art technology.

With a system configuration where the sheath is not introduced into the artery and the conduit 105 is attached to the artery wall, the sheath dimensions can be the same or different as that described above. Similarly, the sheath dimensions may be optimized to have dimensions optimized for the access site to be used (e.g., femoral, radial, brachial, ulnar, subclavian, carotid, etc.) and depending on the indication (e.g., carotid stenting under reverse flow). In some implementations, the conduit 105 from the proximal opening to the distal opening can be about 5 cm in length up to about 30 cm. The coupler 107 can add an additional length resulting in a total length of about 6 cm to about 31 cm. The distal sheath 605 can be shorter than this length, for example between about 5 cm to about 30 cm, so that the distal tip of the distal sheath 605 remains inside the conduit 105 during use. In other implementations, the distal sheath 605 is longer than this length so that at least a portion of the distal sheath 605 extends inside the arterial lumen. The length of the distal sheath 605 extending inside the arterial lumen can be between about 1 cm and about 5 cm. This can depend upon the anatomy of the patient and the length of the conduit 105. In general, when the vessel is about 4-5 cm deep or deeper the insertion of the arterial sheath directly into the vessel faces greater difficulties and challenges. Factors such as the length of the vessel, the presence or absence of disease in the vessel near the desired insertion point, consistency of the vessel wall, operator technique, and the presence of disease distal to a point of insertion can all impact sheath insertion directly into the vessel. The conduit 105 can be used to mitigate challenges that arise due any of these factors. In some implementations, the distal sheath 605 can be inserted about 2.5 cm into the vessel to ensure adequate stability. During insertion of the distal sheath 605, the tip of the dilator can extend beyond the tip of the distal sheath 605 (e.g., about 1.5 cm). In order to provide adequate support for the distal sheath 605 and the dilator during insertion, a guidewire (e.g., 0.035″ guidewire) is also inserted a minimum distance into the vessel beyond the arteriotomy. The distance can be about 5 cm and is preferably further than this. A system that incorporates the conduit 105 can access the vessel and set up reverse flow in a patient without positioning any instrument distal to the arteriotomy, including the distal sheath, a dilator, or guidewire.

The conduit 105 may be a pre-cut conduit. A pre-cut conduit provides an optimal access angle for large bore devices, and eliminates the need for a physician to modify the conduit 105 at the time of the procedure. The conduit 105 may be pre-cut to a desired length, such as about 5 cm in length to about 30 cm as described above. The improved attachment of the conduit 105 to the patient's vessel provides improved sealing of the conduit 105 to the access site, and may allow use of the conduit 105 in other medical specialties that are not as familiar with conduit attachment. In some embodiments, the conduit 105 may be formed of a bio-absorbable material, which facilitates re-access by leaving no material behind at the access site, and a radiopaque marker which allows visualization of the previous access site. In some embodiments, the conduit 105 may be directly attached to the artery or vascular structure via suture or other suitable attachment method (e.g. clips, staples, etc.). In some implementations, the conduit 105 may have a 15°-25° angle at the distal end 1125 of the conduit 105, which can aid attachment of the conduit 105 and direct angle of access to ancillary devices including sheaths and delivery systems. In some embodiments, the conduit 105 may have pre-attached absorbent material, configured as, for example, pledgets or a sewing cuff for attachment to the artery. bio-absorbable materials (e.g. PGA, PGA-PLA, etc.) In some embodiments, the exterior body of the conduit 105 may be configured to be straight, corrugated, or to have external rings for kink resistance. In some embodiments, the conduit 105 may have an embedded radiopaque marker to aid in visualization for re-access. The radiopaque marker may include any substance capable of absorbing X-rays, such as Barium and Iodine. The radiopaque marker may be embedded into the material of the conduit 105, imprinted onto a surface of the conduit 105, adhered to a surface of the conduit, or otherwise positioned on or within the conduit 105 such as to aid in visualization of the access site for potential re-access.

Again with respect to FIGS. 5A-5B, a flush line 635 can be connected to the side of the hemostasis valve 625 and can have a stopcock 640 at its proximal or remote end. The flush-line 635 allows for the introduction of saline, contrast fluid, or the like, during the procedures. The flush line 635 can also allow pressure monitoring during the procedure. A dilator 645 having a tapered distal end 650 can be provided to facilitate introduction of the distal sheath 605 into the common carotid artery. The dilator 645 can be introduced through the hemostasis valve 625 so that the tapered distal end 650 extends through the distal end of the sheath 605. The dilator 645 can have a central lumen to accommodate a guide wire 611. Typically, the guide wire 611 is placed first into the vessel, and the dilator/sheath combination travels over the guide wire 611 as it is being introduced into the vessel. When used with a conduit 105, no guidewire 611 is necessary. Rather, the sheath 605 with the dilator 645 can be inserted through the coupler 107 of the conduit 105 attached to the vessel.

The distal sheath 605 can be configured to establish a curved transition from a generally anterior-posterior approach over the vessel, such as the common carotid artery, to a generally axial luminal direction within the vessel. Arterial access through the vessel wall either from a direct surgical cut down or a percutaneous access may involve different angles of access that are larger than other sites of arterial access, particularly where the insertion site is much closer to the treatment site (i.e., carotid bifurcation) than from other access points. For example, arterial access through the CCA wall may require a larger angle of access compared to access through the wall of the femoral vessel in the groin. In the case of the CCA access and a carotid treatment site, a larger access angle is needed to increase the distance from the insertion site to the treatment site to allow the sheath to be inserted at an adequate distance without the sheath distal tip reaching the carotid bifurcation. For example, the sheath insertion angle is typically 30-45 degrees or even larger via a transcarotid access, whereas the sheath insertion angle may be 15-20 degrees for access into a femoral artery. The sheath can be designed to take a greater bend than is typical with introducer sheaths, without kinking and without causing undue force on the opposing arterial wall. In addition, the sheath tip desirably does not be abut or contact the arterial wall after insertion in a manner that would restrict flow into the sheath. The sheath insertion angle is defined as the angle between the luminal axis of the artery and the longitudinal axis of the sheath.

The sheath body 605 can be formed in a variety of ways to allow for this greater bend required by the angle of access. For example, the sheath and/or the dilator may have a combined flexible bending stiffness less than typical introducer sheaths. In an embodiment, the sheath/dilator combination (i.e., the sheath with the dilator positioned inside the sheath) has a combined flexible stiffness (E*I) in the range of about 80 and 100 N-m²×10⁻⁶, where E is the elastic modulus and I is the area moment of inertia of the device. The sheath alone may have a bending stiffness in the range of about 30 to 40 N-m²×10⁻⁶ and the dilator alone has a bending stiffness in the range of about 40 to 60 N-m²×10⁻⁶. Typical sheath/dilator bending stiffnesses are in the range of 150 to 250 N-m²×10⁻⁶. The greater flexibility may be achieved through choice of materials or design of the reinforcement. For example, the sheath may have a ribbon coil reinforcement of stainless steel with dimensions 0.002″ to 0.003″ thick and 0.005″ to 0.015″ width, and with outer jacket durometer of between 40 and 55 D. In an embodiment, the coil ribbon is 0.003″×0.010″, and the outer jacket durometer is 45 D. In an embodiment, the sheath 605 can be pre-shaped to have a curve or an angle some set distance from the tip, typically 0.5 to 1 cm. The pre-shaped curve or angle can typically provide for a turn in the range from 5° to 90°, preferably from 10° to 30°. For initial introduction, the sheath 605 can be straightened with an obturator or other straight or shaped instrument such as the dilator 645 placed into its lumen. After the sheath 605 has been at least partially introduced through the percutaneous or other arterial wall penetration, the obturator can be withdrawn to allow the sheath 605 to reassume its pre-shaped configuration into the arterial lumen. To retain the curved or angled shape of the sheath body after having been straightened during insertion, the sheath may be heat set in the angled or curved shape during manufacture. Alternately, the reinforcement structure may be constructed out of nitinol and heat shaped into the curved or angled shape during manufacture. Alternately, an additional spring element may be added to the sheath body, for example a strip of spring steel or nitinol, with the correct shape, added to the reinforcement layer of the sheath.

Other sheath configurations include having a deflection mechanism such that the sheath can be placed and the catheter can be deflected in situ to the desired deployment angle. In still other configurations, the catheter has a non-rigid configuration when placed into the lumen of the common carotid artery. Once in place, a pull wire or other stiffening mechanism can be deployed in order to shape and stiffen the sheath into its desired configuration. One particular example of such a mechanism is commonly known as “shape-lock” mechanisms as well described in medical and patent literature.

Another sheath configuration includes a curved dilator inserted into a straight but flexible sheath, so that the dilator and sheath are curved during insertion. The sheath is flexible enough to conform to the anatomy after dilator removal.

Another sheath embodiment is a sheath that includes one or more flexible distal sections, such that once inserted and in the angled configuration, the sheath is able to bend at a large angle without kinking and without causing undue force on the opposing arterial wall. In one embodiment, there is a distal-most section of sheath body 605 which is more flexible than the remainder of the sheath body. For example, the flexural stiffness of the distal-most section is one half to one tenth the flexural stiffness of the remainder of the sheath body 605. In an embodiment, the distal-most section has a flexural stiffness in the range 30 to 300 N-mm² and the remaining portion of the sheath body 605 has a flexural stiffness in the range 500 to 1500 N-mm². For a sheath configured for a CCA access site, the flexible, distal most section includes a significant portion of the sheath body 605 which may be expressed as a ratio. In an embodiment, the ratio of length of the flexible, distal-most section to the overall length of the sheath body 605 is at least one tenth and at most one half the length of the entire sheath body 605. This change in flexibility may be achieved by various methods. For example, the outer jacket may change in durometer and/or material at various sections. Alternately, the reinforcement structure or the materials may change over the length of the sheath body. In an embodiment, the distal-most flexible section ranges from 1 cm to 3 cm. In an embodiment with more than one flexible section, a less flexible section (with respect to the distal-most section) may be 1 cm to 2 cm from the distal-most proximal section. In an embodiment, the distal flexible section has a bending stiffness in the range of about 30 to 50 N-m²×10⁻⁶ and the less flexible section has a bending stiffness in the range of about 50 and 100 N-m²×10⁻⁶. In another embodiment, a more flexible section is located between 0.5 and 1.5 cm for a length of between 1 and 2 cm, to create an articulating section that allows the distal section of the sheath to align more easily with the vessel axis though the sheath enters the artery at an angle. These configurations with variable flexibility sections may be manufactured in several manners. For example the reinforced, less flexible section may vary such that there is stiffer reinforcement in the proximal section and more flexible reinforcement in the distal section or in the articulating section. In an embodiment, an outer-most jacket material of the sheath is 45 D to 70 D durometer in the proximal section and 80 A to 25 D in the distal-most section. In an embodiment, the flexibility of the sheath varies continuously along the length of the sheath body. The flexible distal section of the sheath body 605 allows the sheath to bend and the distal tip to be in general alignment with the vessel lumen. In an embodiment, the distal section is made with a more flexible reinforcement structure, either by varying the pitch of a coil or braid or by incorporating a cut hypotube with differing cut patterns. Alternately the distal section has a different reinforcement structure than the proximal section.

In an embodiment, the distal sheath tapered tip is manufactured from harder material than the distal sheath body. A purpose of this is to facilitate ease of sheath insertion by allowing for a very smooth taper on the sheath and reduce the change of sheath tip distortion or ovalizing during and after sheath insertion into the vessel. In one example the distal tapered tip material is manufactured from a higher durometer material, for example a 60-72 D shore material. In another example, distal tip is manufactured from a separate material, for example HDPE, stainless steel, or other suitable polymers or metals. In an additional embodiment, the distal tip is manufactured from radiopaque material, either as an additive to the polymer material, for example tungsten or barium sulfate, or as an inherent property of the material (as is the case with most metal materials).

When used with the conduit 105, the sheath need not be capable of such angles because the sheath can remain within the conduit lumen rather than passing distal to the arteriotomy and inserting directly into the vessel lumen.

In another embodiment, the introducer guide wire 611 is optimally configured for transcarotid access. Typically when inserting an introducer sheath into a vessel, an introducer guide wire is first inserted into the vessel. This may be done either with a micropuncture technique or a modified Seldinger technique. Usually there is a long length of vessel in the direction that the sheath is to be inserted into which an introducer guidewire may be inserted, for example into the femoral artery. In this instance, a user may introduce a guide wire between 10 and 15 cm or more into the vessel before inserting the sheath. The guide wire is designed to have a flexible distal section so as not to injure the vessel when being introduced into the artery. The flexible section of an introducer sheath guide wire 611 is typically 5 to 6 cm in length, with a gradual transition to the stiffer section. Inserting the guide wire 10 to 15 cm means the stiffer section of the guide wire is positioned in the area of the puncture and allows a stable support for subsequent insertion of the sheath and dilator into the vessel. However, in the case of transcarotid sheath insertion into the common carotid artery, there is a limit on how much guide wire may be inserted into the carotid artery. In cases with carotid artery disease at the bifurcation or in the internal carotid artery, it is desirable to minimize the risk of emboli by inserting the wire into the external carotid artery (ECA), which would mean only about 5 to 7 cm of guide wire insertion, or to stop it before it reaches the bifurcation, which would be only 3 to 5 cm of guide wire insertion. Thus, a transcarotid sheath guidewire may have a distal flexible section of between 3 and 4 cm, and/or a shorter transition to a stiffer section. Alternately, a transcarotid sheath guidewire has an atraumatic tip section but have a very distal and short transition to a stiffer section. For example, the soft tip section is 1.5 to 2.5 cm, followed by a transition section with length from 3 to 5 cm, followed by a stiffer proximal segment, with the stiffer proximal section comprising the remainder of the wire.

To use this sheath system embodiment, a micropuncture kit can be used for initial access to the vessel. The 0.018″ guide wire is first inserted into the vessel through a 22 Ga needle. The sheath system which is coaxially assembled is inserted over the 0.018″ wire. The inner tube is first advanced over the 0.018″ wire which essentially transforms it into the equivalent of an 0.035″ or 0.038″ guide wire in both outer diameter and mechanical support. It is locked down to the 0.018″ wire on the proximal end. The sheath and dilator are then advanced over the 0.018″ wire and inner tube into the vessel. This configuration eliminates the wire exchange step without the need for a longer dilator taper as with current transradial sheaths and with the same guide wire support as standard introducer sheaths. As described above, this configuration of sheath system may include stopper features which prevent inadvertent advancement too far of the 0.018″ guide wire and/or inner tube during sheath insertion. Once the sheath is inserted, the dilator, inner tube, and 0.018″ guide wire are removed.

An operator also can create a large hole in the artery wall and attach the conduit 105 to provide a large access portion through which a sheath may be delivered or for direct attachment of a flow reversal system without a sheath.

Another arterial access device is shown in FIGS. 5D-5E. This configuration has a different style of connection to the flow shunt than the versions described previously with respect to FIGS. 5A-5B. FIG. 5D shows the components of the arterial access device 110 including arterial access sheath 605, sheath dilator 645, conduit 105, and sheath guidewire 611. FIG. 5E shows the arterial access device 110 as it would be assembled for insertion over the sheath guide wire 611 into the vessel, which can be the common carotid artery or another access site as described elsewhere herein. After the sheath is inserted to be in fluid communication with the artery, such as via the conduit 105, the sheath guide wire 611 and sheath dilator 645, if used, are removed. In this configuration, the sheath has a sheath body 605, proximal extension 610, and proximal hemostasis valve 625 with flush line 635 and stopcock 640. The proximal extension 610 extends from a Y-adapter 660 to the hemostasis valve 625 where the flush line 635 is connected. The sheath body 605 is the portion that is sized to be inserted into the carotid artery and is actually inserted into the artery during use.

Instead of a Y-connector with a flow line connection terminating in a valve, the sheath has a Y-adaptor 660 that connects the distal portion of the sheath to the proximal extension 610. The Y-adapter can also include a valve 670 that can be operated to open and close fluid connection to a connector or hub 680 that can be removably connected to a flow line such as a shunt. The valve 670 is positioned immediately adjacent to an internal lumen of the adapter 660, which communicates with the internal lumen of the sheath body 605. The valve 670 is closed to the connector during prep of the arterial sheath. The valve 670 is configured so that there is no potential for trapped air during prep of the sheath. The valve 670 is open to the connector once the flow shunt 120 is connected to hub 680, and would allow blood flow from the arterial sheath into the shunt. This configuration eliminates the need to prep both a flush line and flow line, instead allowing prep from the single flush line 635 and stopcock 640. This single-point prep is identical to prep of conventional introducer sheaths which do not have connections to shunt lines, and is therefore more familiar and convenient to the user. In addition, the lack of flow line on the sheath makes handling of the arterial sheath easier during prep and insertion into the artery.

With reference again to FIG. 5D, the sheath may also contain a second more distal connector 690, which is separated from the Y-adaptor 660 by a segment of tubing 665. A purpose of this second connector and the tubing 665 is to allow the valve 670 to be positioned further proximal from the distal tip of the sheath, while still limiting the length of the insertable portion of the sheath 605, and therefore allow a reduced level of exposure of the user to the radiation source as the flow shunt is connected to the arterial sheath during the procedure. In an embodiment, the distal connector 690 contains suture eyelets to aid in securement of the sheath to the patient once positioned.

As an example method, the conduit 105 can be useful during a transcarotid artery revascularization (TCAR) procedure. In this procedure, the arterial sheath 605 can be inserted into the common carotid artery (CCA) of the patient, either directly or through the conduit 105. It should be appreciated that the sheath 605 need not insert through the conduit 105 so far as to also insert into the common carotid artery. As described elsewhere herein, to achieve reverse flow of blood, the CCA may be occluded to stop antegrade blood flow from the aorta through the CCA. Flow through the CCA can be occluded with an external vessel loop or tape, a vascular clamp, an internal occlusion member such as a balloon, or other type of occlusion means such as the tourniquet 2105 shown in FIG. 1A. When flow through CCA is blocked, the natural pressure gradient between the internal carotid artery (ICA) and the venous system will cause blood to flow in a retrograde or reverse direction from the cerebral vasculature. Blood from the ICA and the external carotid artery (ECA) flows in a retrograde direction and the systems described herein allow the retrograde blood to flow into the sheath 605, through the flow controller 125, the venous return device 115, and then returned into the patient's femoral vein as described elsewhere herein. Loose embolic material can be carried with the retrograde blood flow into the arterial sheath 605.

Manual occlusion of the vessel V by a clinician at an occlusion location proximal to the distal tip of the sheath 605 may be provided from the outside of the vessel V using a vascular clamp 800, such as a Rummel tourniquet or vessel loop positioned proximal to the sheath insertion site. An occlusion device may fit externally to the vessel V around the sheath tip, for example, an elastic loop, inflatable cuff, or a mechanical clamp that can be tightened around the vessel and the distal sheath tip. In a flow reversal system, this type of vessel occlusion can minimize creation of a zone of static blood flow, thereby reducing the risk of thrombus formation. This also ensures that the sheath tip is axially aligned with the vessel V and avoids partially or fully blocking the distal opening by the vessel wall.

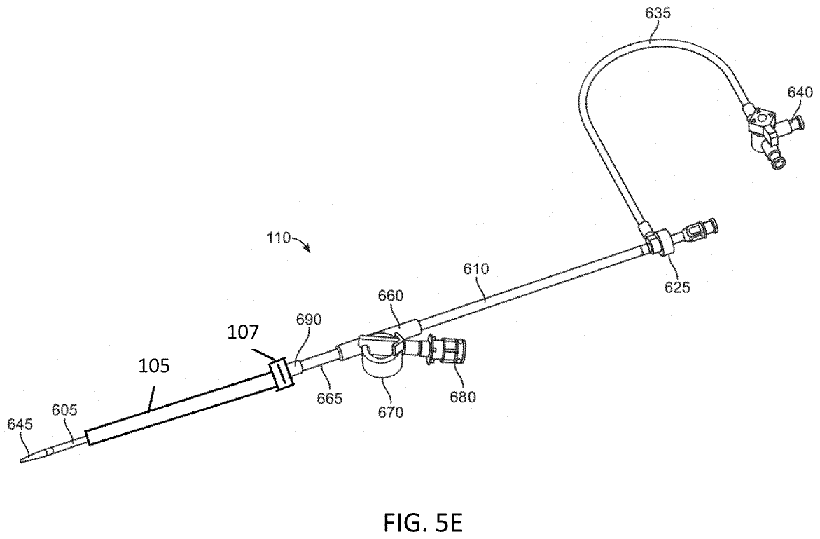

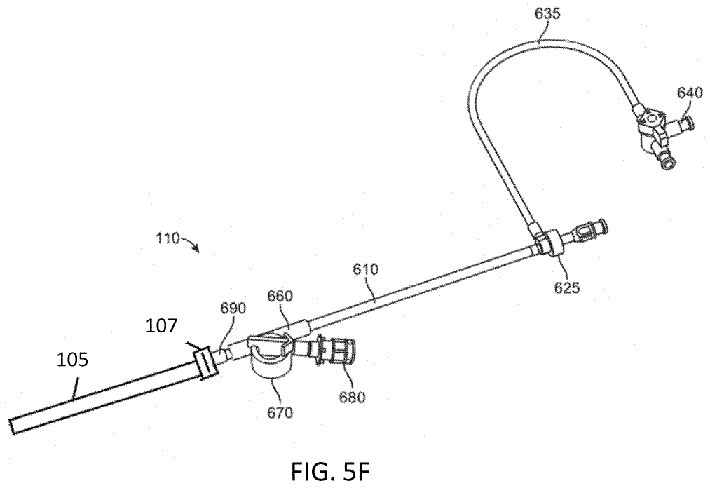

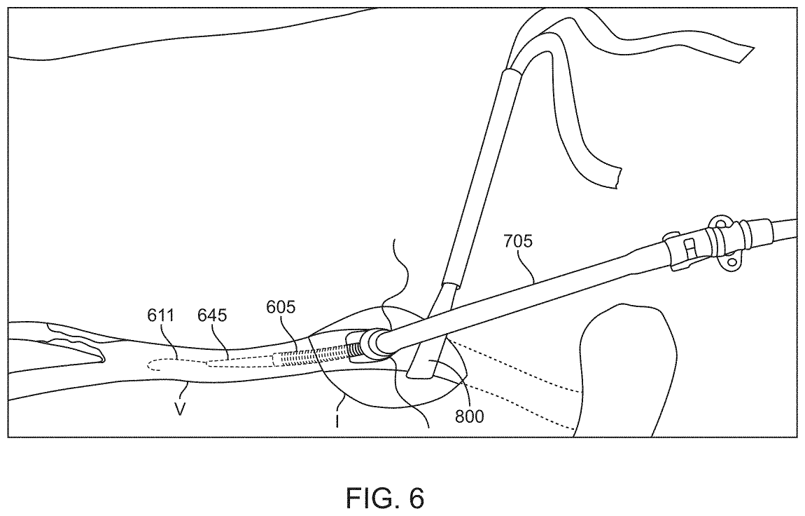

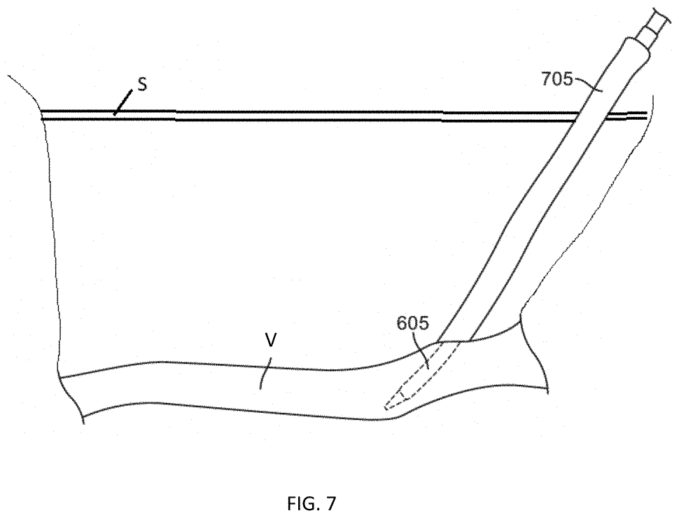

FIG. 6 illustrates an arterial sheath 605 inserted directly within a vessel V, such as the common carotid artery, or another vascular access site, exposed through an incision I. The vascular access site need not be a cut-down procedure and can also be percutaneous. A sheath guidewire 611 and an arterial access sheath dilator 645 protrude out from a distal opening near the distal region of the sheath 605. A sheath stopper 705 can be used with the sheath 605 to prevent over-insertion of the distal arterial sheath 605 through the arteriotomy, as described U.S. Publication No. 2019/0125512, which is incorporated by reference here. FIG. 7 shows the arterial sheath 605 extending distal to the sheath stopper 705 inside of a vessel V. The conduit 105 can be used to insert an arterial sheath 605 into a vessel without any sheath stopper 705 or mechanical hard-stop that establishes an insertion length of the sheath 605, for example, where deeper access of the sheath 605 is desired. The sheath 605 may be advanced through the common carotid artery distal to the bifurcation into the distal portion of the internal carotid artery or past the petrous portion of the ICA. In this implementation, it may be preferred to exclude a sheath stopper 705 to provide deeper access that is not limited by a mechanical stop. Other indications may benefit from the presence of the sheath stopper, for example, where the sheath is preferably implanted proximal to the bifurcation.

The sheath stopper 705 can be in the form of a tube that is coaxially received over the exterior of the distal sheath 605. The sheath stopper 705 is configured to prevent the sheath from being inserted too far into the vessel V. The sheath stopper 705 is sized and shaped to be positioned over the sheath body 605 such that it covers a portion of the sheath body 605 and leaves a distal portion of the sheath body 605 exposed. The sheath stopper 705 may have a flared proximal end that engages the adapter 620, and a distal end that abuts against the exterior of the vessel V. The length of the sheath stopper 705 limits the introduction of the sheath 605 to the exposed distal portion of the sheath 605 such that the sheath insertion length is limited to the exposed distal portion of the sheath. In an embodiment, the sheath stopper limits the exposed distal portion to a range between 2 and 3 cm. In an embodiment, the sheath stopper limited the exposed distal portion to 2.5 cm. In other words, the sheath stopper may limit insertion of the sheath into the artery to a range between about 2 and 3 cm or to 2.5 cm. Second, the sheath stopper 705 can engage a pre-deployed puncture closure device disposed in the carotid artery wall, if present, to permit the sheath 605 to be withdrawn without dislodging the closure device. The sheath stopper 705 may also be made from flexible material, or the sheath stopper 705 include articulating or sections of increased flexibility so that it allows the sheath to bend as needed in a proper position once inserted into the artery. The sheath stopper may be plastically bendable such that it can be bent into a desired shape such that it retains the shape when released by a user. The distal portion of the sheath stopper may be made from stiffer material, and the proximal portion may be made from more flexible material. In an embodiment, the stiffer material is 85 A durometer and the more flexible section is 50 A durometer. In an embodiment, the stiffer distal portion is 1 to 4 cm of the sheath stopper 705. The sheath stopper 705 may be removable from the sheath so that if the user desired a greater length of sheath insertion, the user could remove the sheath stopper 705, cut the length (of the sheath stopper) shorter, and re-assemble the sheath stopper 705 onto the sheath such that a greater length of insertable sheath length protrudes from the sheath stopper 705.

The sheath stopper 705 may be deformed from a first shape, such as a straight shape, into a second shape different from the first shape wherein the sheath stopper retains the second shape until a sufficient external force acts on the sheath stopper to change its shape. The second shape may be for example non-straight, curved, or an otherwise contoured or irregular shape. The sheath stopper 705 may have multiple bends as well as straight sections. The sheath stopper 705 has a greater stiffness than the sheath 605 such that the sheath 605 takes on a shape or contour that conforms to the shape of contour of the sheath stopper 705.

The sheath stopper 705 may be shaped according to an angle of the sheath insertion into the artery and the depth of the artery or body habitus of the patient. This feature reduces the force of the sheath tip in the blood vessel wall, especially in cases where the sheath is inserted at a steep angle into the vessel. The sheath stopper may be bent or otherwise deformed into a shape that assists in orienting the sheath coaxially with the artery being entered even if the angle of the entry into the arterial incision is relatively steep. The sheath stopper may be shaped by an operator prior to sheath insertion into the patient. Or, the sheath stopper may be shaped and/or re-shaped in situ after the sheath has been inserted into the artery.

In an embodiment, the sheath stopper 705 is made from malleable material, or with an integral malleable component positioned on or in the sheath stopper. In another embodiment, the sheath stopper is constructed to be articulated using an actuator such as concentric tubes, pull wires, or the like. The wall of the sheath stopper may be reinforced with a ductile wire or ribbon to assist it in holding its shape against external forces such as when the sheath stopper encounters an arterial or entryway bend. Or the sheath stopper may be constructed of a homogeneous malleable tube material, including metal and polymer. The sheath stopper body may also be at least partially constructed of a reinforced braid or coil capable of retaining its shape after deformation.

Another sheath stopper embodiment is configured to facilitate adjustment of the sheath stopper position (relative to the sheath) even after the sheath is positioned in the vessel. One embodiment of the sheath stopper includes a tube with a slit along most or all of the length, so that the sheath stopper can be peeled away from the sheath body, moved forward or backwards as desired, and then re-positioned along the length of the sheath body. The tube may have a tab or feature on the proximal end so it may be grasped and more easily to peel away.

In another embodiment, the sheath stopper is a very short tube (such as a band), or ring that resides on the distal section of the sheath body. The sheath stopper may include a feature that could be grasped easily by forceps, for example, and pulled back or forwards into a new position as desired to set the sheath insertion length to be appropriate for the procedure. The sheath stopper may be fixed to the sheath body through either friction from the tube material, or a clamp that can be opened or closed against the sheath body. The clamp may be a spring-loaded clamp that is normally clamped onto the sheath body. To move the sheath stopper, the user may open the clamp with his or her fingers or an instrument, adjust the position of the clamp, and then release the clamp. The clamp is designed not to interfere with the body of the sheath.

In another embodiment, the sheath stopper includes a feature that allows suturing the sheath stopper and sheath to the tissue of the patient, to improve securement of the sheath and reduce risk of sheath dislodgement. The feature may be suture eyelets that are attached or molded into the sheath stopper tube.

In another embodiment, as shown in FIG. 6, the sheath stopper 705 includes a distal flange sized and shaped to distribute the force of the sheath stopper over a larger area on the vessel wall and thereby reduce the risk of vessel injury or accidental insertion of the sheath stopper through the arteriotomy and into the vessel. The flange may have a rounded shape or other atraumatic shape that is sufficiently large to distribute the force of the sheath stopper over a large area on the vessel wall. In an embodiment, the flange is inflatable or mechanically expandable. For example, the arterial sheath and sheath stopper may be inserted through a small puncture in the skin into the surgical area, and then expanded prior to insertion of the sheath into the artery.

The sheath stopper may include one or more cutouts or indents along the length of the sheath stopper which are patterned in a staggered configuration such that the indents increase the bendability of the sheath stopper while maintaining axial strength to allow forward force of the sheath stopper against the arterial wall. The indents may also be used to facilitate securement of the sheath to the patient via sutures, to mitigate against sheath dislodgement. The sheath stopper may also include a connector element on the proximal end which corresponds to features on the arterial sheath such that the sheath stopper can be locked or unlocked from the arterial sheath. For example, the connector element is a hub with generally L-shaped slots that correspond to pins on the hub to create a bayonet mount-style connection. In this manner, the sheath stopper can be securely attached to the hub to reduce the likelihood that the sheath stopper will be inadvertently removed from the hub unless it is unlocked from the hub.

FIG. 7 shows the distal end of the arterial sheath 605 extending inside the vessel V through the sheath stopper 705 along an angle of approach that is very steep. The angle between the axis of the vessel V and the axis of the sheath 605 (proximal to the bend) is shown as being approximately 50-60 degrees, but in some patient anatomies this angle can be closer to 90 degrees from the axis of the vessel V. The distal-most tip of the sheath 605 is extending towards the posterior wall of the vessel V, which along with the steep angle increases the risk of arterial dissection and perforation.

The conduit 105, unlike the sheath stopper 705, can have a distal-most end configured to be affixed to a wall of the vessel via suturing creating a leak-free connection with the vessel. The flexible conduit 105 can effectively become an extension of the vessel itself like a temporary anastomosis through which the arterial sheath 605 can be inserted.

The material of the conduit 105 as described elsewhere herein is inert, biocompatible, and strong. The material of the conduit 105 can be conformable, customizable, and relatively easy-to-handle. Preferably, the material of the conduit 105 is able to be attached to the vessel wall by sewing as opposed to another type of conduit 105 that is only able to be inserted through a hole in the side wall of the vessel. In some implementations, the conduit 105 may be formed of polyethylene terephthalate (Dacron), a polymer such as polyfluorotetraethylene (PTFE), or bio-absorbable materials (e.g. PGA, PGA-PLA, PLGA, PCL, etc.). The conduit 105 can be formed of one or more layers of a material such as expanded PTFE (ePTFE). Soft, flexible materials like ePTFE that can also carry fluids such as blood with minimal to no leakage across the wall are preferred. The softness and compliance of the material used to form the conduit 105 allows it to be sewn with relative ease because a needle and suture may be passed through the wall. Also, the material is preferably resistant to tearing, which also makes it more easily sewn than for example a silicone rubber tube. The conduit 105 is preferably formed of a compliant material that has a resistance to tearing and that is relatively impervious to fluids and thrombo-resistant so it is capable of carrying blood through its lumen and also capable of creating a leak-free seal at the conduit-vessel interface. ePTFE has all of these characteristics making it particularly suitable to form the conduit 105. Other materials can have similar properties, such as tightly woven Dacron. A flexible elastomeric material, such as silicone, could potentially be used if the conduit incorporates additional structural reinforcement to prevent tearing. However, a reinforced silicone elastomer provides a more cost-effective alternative to ePTFE considering the short-termed nature of the temporarily attached conduit 105.

The layer(s) of the conduit 105 can be formed into a substantially cylindrical or tubular shape according to known techniques (e.g., extruding, dip-coating a mandrel, and the like). The tubular shape can be tapered so that a first end of the conduit 105 is smaller than an opposite end of the conduit 105. The inner diameter of the conduit 105 can be constant from a proximal to a distal end or can increase proximally near the coupler.

The conduit 105 can be designed for suturing to the vessel wall. The material of the tubular conduit 105 is configured to be penetrated by a needle and suture without snagging, tearing, or ripping a wall of the conduit 105 and also while maintaining a substantial seal. The wall thickness of the conduit 105 can be about 0.25 mm to about 3.0 mm. In some implementations, the conduit 105 has a graduated wall thickness such that at least one region is reduced in wall thickness for suturing. The reduced wall thickness segment of the conduit 105 can be at least 1 mm long from a distal-most end. The seal of the sutured region allows for the conduit 105 to be used to pass fluids to and from the vessel lumen as well as insert devices through it.

In some implementations, the conduit 105 can include an inner layer, a middle layer, and an outer layer of a material. The inner layer can be heparinized to prevent platelet adherence and the middle layer can be elastomeric and configured to minimize weeping and suture hole bleeding following suturing.

The external surface of the conduit 105 can have one or more visual markers to identify lengths of the conduit 105 from the proximal coupler. The visual markers can provide a guide to a user attempting to customize a length of the conduit 105 prior to use. For example, the conduit 105 can be provided in a length of at least about 20 cm from coupler to distal-most end. The conduit 105 can have a visual marker every 5 cm from the coupler to the distal-most end such that the 20 cm length of the conduit 105 can be modified more easily to 15 cm, 10 cm, or 5 cm in length. The visual markers can provide any of a variety of graduations to identify lengths.

FIGS. 8A-8C show a sheath 605 extending through a coupler 107 of a conduit 105 having a distal end attached to the wall of the vessel V. The sheath 605 can insert through the coupler 107 on a proximal end of the conduit 105. FIG. 8A shows the distal end of the sheath 605 can extend completely through the conduit 105 and out the distal end so that distal end of the sheath 605 is positioned inside the vessel V. FIG. 8B shows the distal end of the sheath 605 remains inside the lumen of the conduit 105 and does not enter the vessel V lumen. In this implementation, the distal end of the sheath 605 stays out of the vessel and the risk of dissection or perforation is reduced. In still further implementations, the arterial access device has no distal sheath 605 inside of the conduit 105 (see FIG. 5F) and the coupler 107 on the proximal end of the conduit 105 operatively couples with the distal connector 690 of the arterial access device directly. The lumen of the conduit 105 maintains its maximum size without any flow resistance due to the presence of a smaller tube within its lumen.

FIG. 8C shows the conduit 105 coupled directly to a vessel and having a coupler 107 on a proximal end that can couple to an arterial access device 110 of a reverse flow system. As discussed elsewhere herein, the conduit 105 can be part of a retrograde flow shunt circuit without any sheath extending within its lumen. The conduit 105 can therefore take the place of the access sheath and connect to the reverse flow circuit directly and also be configured to insert interventional devices into the vessel without use of a conventional access sheath (e.g., 8 Fr sheath). The coupler 107 of the conduit 105 can be a simple luer fitting (see FIG. 5C) or the coupler 107 can be a multi-port device having rotating hemostatic valves 670 at each port (see FIG. 8C). Any of a variety of hemostatic coupling configurations are considered herein to provide fluid communication between the reverse flow shunt circuit and the lumen of the conduit 105 so that reverse flow through the CCA can be directed out through the system 110 and so that one or more devices or fluids can be provided to the CCA and beyond.

The hemostatic valve 670 (e.g. Tuohy Valve, passive valve) may be configured as an integrated hub or hemostatic valve 670. The hemostatic valve 670 provides hemostasis without the need for insertion of a specific sheath into the conduit 105. The integrated hub or hemostatic valve 670 can also provide hemostasis during insertion and removal of large bore tools in concert with a clamp. A primary port with an insert for small bore access, or small bore adaptor 1155, can be used to increase the utility of the assembly for small bore applications. A secondary port, such as a Y-port 1120, may provide additional opportunities for reverse blood flow or use of other surgical tools. The integrated hub or hemostatic valve 670 may be attached to the conduit 105 directly. The integrated hub or hemostatic valve 670 may be attached to the distal tubular body 1605 via an adhesive and/or over molding. The integrated hub or hemostatic valve 670 may be a passive hemostasis valve with a self-sealing polymer valve(s). The integrated hub or hemostatic valve 670 may be an active Tuohy valve, such as to allow passage of delivery devices and/or sheath(es) having the potential ability to limit blood flow. The integrated hub or hemostatic valve 670 may be a combination of active and/or passive valves. The integrated hub or hemostatic valve 670 may be silicone coated, or coated with another suitable polymer or plastic. The integrated hub or hemostatic valve 670 may include a second port 1120, such as for purging, deairing, reverse flow, or other applications. The integrated hub or hemostatic valve 670 may have an insert, for example a hubbed insert or small bore adapter 1155, such as to facilitate small bore access as with a sheath/pigtail and wire delivery. The second port 1120 may be configured as a “Y” port.

Venous Return Device

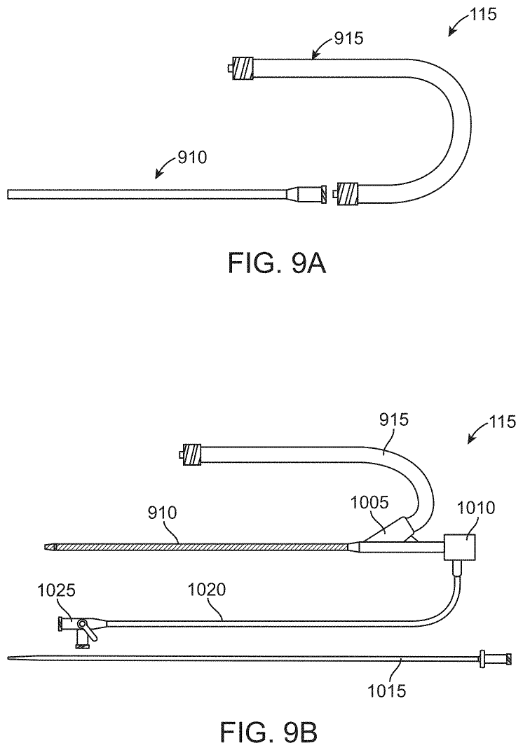

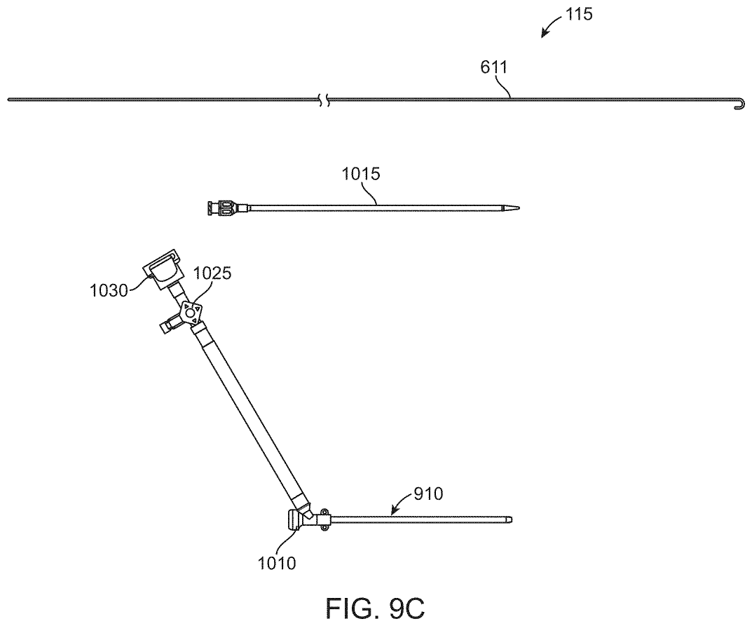

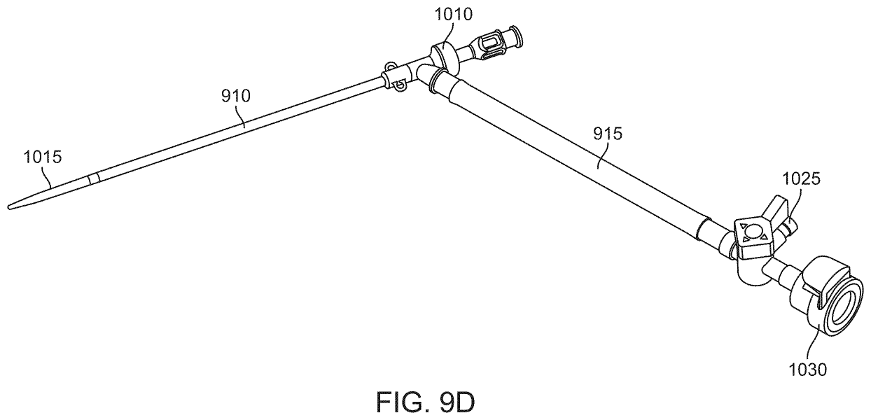

Referring now to FIGS. 9A-9D, the venous return device 115 can include a distal sheath 910 and a flow line 915, which connects to and forms a leg of the shunt 120 when the system is in use. The distal sheath 910 is adapted to be introduced through an incision or puncture into a venous return location, such as the jugular vein or femoral vein. The distal sheath 910 and flow line 915 can be permanently affixed, or can be attached using a conventional luer fitting, as shown in FIG. 9A. Optionally, as shown in FIG. 9B, the sheath 910 can be joined to the flow line 915 by a Y-connector 1005. The Y-connector 1005 can include a hemostasis valve 1010. The venous return device also include a venous sheath dilator 1015 and an introducer guide wire 611 to facilitate introduction of the venous return device into the internal jugular vein or other vein. As with the arterial access dilator 645, the venous dilator 1015 includes a central guide wire lumen so the venous sheath and dilator combination can be placed over the guide wire 611. Optionally, the venous sheath 910 can include a flush line 1020 with a stopcock 1025 at its proximal or remote end.

An alternate configuration is shown in FIGS. 9C and 9D. FIG. 9C shows the components of the venous return device 115 including venous return sheath 910, sheath dilator 1015, and sheath guidewire 611. FIG. 9D shows the venous return device 115 as it would be assembled for insertion over the sheath guide wire 611 into a central vein. Once the sheath is inserted into the vein, the dilator and guidewire are removed. The venous sheath can include a hemostasis valve 1010 and flow line 915. A stopcock 1025 on the end of the flow line allows the venous sheath to be flushed via the flow line prior to use. This configuration allows the sheath to be prepped from a single point, as is done with conventional introducer sheaths. Connection to the flow shunt 120 is made with a connector 1030 on the stopcock 1025.

In order to reduce the overall system flow resistance, the arterial access flow line 615 (FIG. 5A) and the venous return flow line 915, and Y-connectors 620 (FIG. 5A) and 1005 (FIG. 9B), can each have a relatively large flow lumen inner diameter, typically being in the range from 2.54 mm (0.100 inch) to 5.08 mm (0.200 inch), and a relatively short length, typically being in the range from 10 cm to 20 cm. The low system flow resistance is desirable since it permits the flow to be maximized during portions of a procedure when the risk of emboli is at its greatest. The low system flow resistance also allows the use of a variable flow resistance for controlling flow in the system, as described in more detail below. The dimensions of the venous return sheath 910 can be generally the same as those described for the arterial access sheath 605 above. In the venous return sheath, an extension for the hemostasis valve 1010 is not required.

Retrograde Shunt

The shunt 120 can be formed of a single tube or multiple, connected tubes that provide fluid communication between the arterial access catheter 110 and the venous return catheter 115 to provide a pathway for retrograde blood flow therebetween. As shown in FIG. 1A, the shunt 120 connects at one end (via connector 127a) to the flow line 615 of the arterial access device 110, and at an opposite end (via connector 127b) to the flow line 915 of the venous return catheter 115.

In an embodiment, the shunt 120 can be formed of at least one tube that communicates with the flow control assembly 125. The shunt 120 can be any structure that provides a fluid pathway for blood flow. The shunt 120 can have a single lumen or it can have multiple lumens. The shunt 120 can be removably attached to the flow control assembly 125, conduit 105, arterial access device 110, and/or venous return device 115. Prior to use, the user can select a shunt 120 with a length that is most appropriate for use with the arterial access location and venous return location. In an embodiment, the shunt 120 can include one or more extension tubes that can be used to vary the length of the shunt 120. The extension tubes can be modularly attached to the shunt 120 to achieve a desired length. The modular aspect of the shunt 120 permits the user to lengthen the shunt 120 as needed depending on the site of venous return. For example, in some patients, the internal jugular vein IJV is small and/or tortuous. The risk of complications at this site may be higher than at some other locations, due to proximity to other anatomic structures. In addition, hematoma in the neck may lead to airway obstruction and/or cerebral vascular complications. Consequently, for such patients it may be desirable to locate the venous return site at a location other than the internal jugular vein IJV, such as the femoral vein. A femoral vein return site may be accomplished percutaneously, with lower risk of serious complication, and also offers an alternative venous access to the central vein if the internal jugular vein IJV is not available. Furthermore, the femoral venous return changes the layout of the reverse flow shunt such that the shunt controls may be located closer to the “working area” of the intervention, where the devices are being introduced and the contrast injection port is located.

In an embodiment, the shunt 120 has an internal diameter of 4.76 mm ( 3/16 inch) and has a length of 40-70 cm. As mentioned, the length of the shunt can be adjusted. In an embodiment, connectors between the shunt and the arterial and/or venous access devices are configured to minimize flow resistance. In an embodiment, the arterial access sheath 110, the retrograde shunt 120, and the venous return sheath 115 are combined to create a low flow resistance arterio-venous AV shunt, as shown in FIGS. 1A-1D. As described above, the connections and flow lines of all these devices are optimized to minimize or reduce the resistance to flow. In an embodiment, the AV shunt has a flow resistance which enables a flow of up to 300 mL/minute when no device is in the arterial sheath 110 and when the AV shunt is connected to a fluid source with the viscosity of blood and a static pressure head of 60 mmHg. The actual shunt resistance may vary depending on the presence or absence of a check valve or a filter, or the length of the shunt, and may enable a flow of between 150 and 300 mL/min.

When there is a device such as a stent delivery catheter in the arterial sheath, there is a section of the arterial sheath that has increased flow resistance, which in turn increases the flow resistance of the overall AV shunt. This increase in flow resistance has a corresponding reduction in flow. In an embodiment, the Y-arm 620 as shown in FIG. 5A connects the arterial sheath body 605 to the flow line 615 some distance away from the hemostasis valve 625 where the catheter is introduced into the sheath. This distance is set by the length of the proximal extension 610. Thus the section of the arterial sheath that is restricted by the catheter is limited to the length of the sheath body 605. The actual flow restriction will depend on the length and inner diameter of the sheath body 605, and the outer diameter of the catheter. As described above, the sheath body 605 length may range from 5 to 15 cm, usually being from 10 cm to 12 cm, and the inner diameter is typically in the range from 7 Fr (1 Fr=0.33 mm), to 10 Fr, usually being 8 Fr. Stent delivery catheters may range from 3.7 Fr. to 5.0 or 6.0 Fr, depending on the size of the stent and the manufacturer. This restriction may further be reduced if the sheath body is designed to increase in inner diameter for the portion not in the vessel (a stepped sheath body), as shown in FIG. 5B. Since flow restriction is proportional to luminal distances to the fourth power, small increases in luminal or annular areas result in large reductions in flow resistance.

The presence of the conduit 105 further reduces the flow resistance of the system 100, particularly where the conduit 105 is coupled directly to the distal coupler 690 of the arterial access system 110 and the arterial access system 110 lacks a distal sheath 605 or where the conduit 105 is coupled directly to a reverse flow shunt without any arterial access system 110. As discussed elsewhere herein, the inner diameter of the conduit 105 can be very large (i.e. between about 5-6 mm) compared to the inner diameter of an interventional device such as a stent delivery catheter or even an arterial access sheath designed to enter the vessel lumen (e.g., between 2.3 mm and 3.0 mm). This size range for the conduit 105 can facilitate a reasonably quick anastomosis time and provides ample inner diameter for the delivery of devices having a variety of sizes. The conduit 105 significantly reduces the flow resistance through the reverse flow circuit compared to an 8 Fr or even a 10 Fr arterial sheath. The inner dimension of the flow path proximal to the conduit 105 can be enlarged as well resulting in a large increase in the retrograde flow through the system while increasing the safety to a patients presenting with challenging anatomy for sheath insertion.

The flow control assembly and regulation and monitoring of the retrograde flow through the system is described in detail in U.S. Publication No. 2019/0125512, which is incorporated by reference herein.

### Exemplary Methods of Use

Entry to the common carotid artery CCA can be via a transcarotid approach through a surgical cut-down. The conduit 105 can be provided with the coupler 107 integral with the proximal end of the conduit 105 or the coupler 107 can be attached to the proximal end of the conduit 105 at the time of surgery. The conduit 105 can be attached to the CCA using any of a variety of standard surgical techniques for end-to-side colligation or anastomosis as discussed elsewhere herein. The conduit 105 can be prepared by cutting the distal end region to a desired overall length. The cut through the distal end region of the conduit 105 can be at an angle relative to the longitudinal axis of the lumen through the conduit 105, such as an oblique angle thereby forming a toe and a heel at the distal end of the conduit 105. An arteriotomy can be made in the wall of the exposed CCA at a target location creating a hole of a desired size. The length of the arteriotomy can be one and half times the diameter of the conduit 105. In other implementations, an incision in the wall of the vessel can be made and the incision enlarged into a hole. In an implementation, the hole can be about 5 mm to 6 mm along its greatest dimension. The distal end of the conduit 105 can be sutured to the vessel wall encircling the hole using any of a variety of suture techniques to create a sealed connection between the distal end of the conduit 105 and the vessel wall. As mentioned above, the arteriotomy can be formed through the conduit 105 already attached to the vessel wall to provide a bloodless/clampless anastomosis. The sutures for coupling the conduit 105 to the vessel may be pre-placed and/or can incorporate one or more attachment features for facilitating faster anastomosis with decreased bleeding such as a hood, gasket, suture ring, or other distal feature on the conduit.

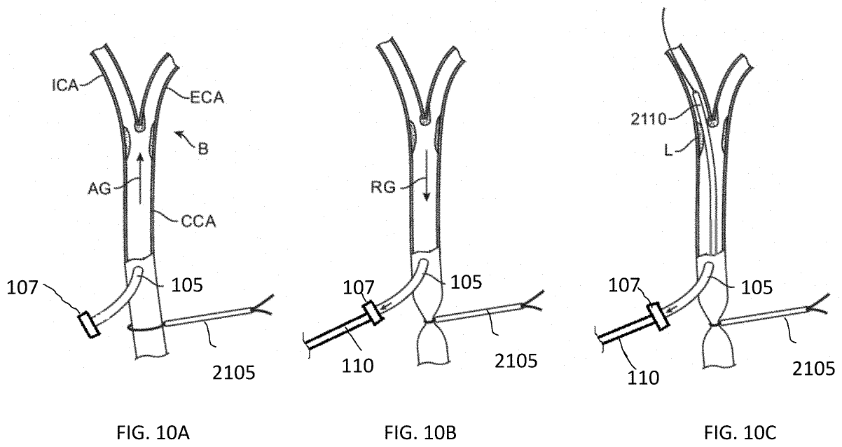

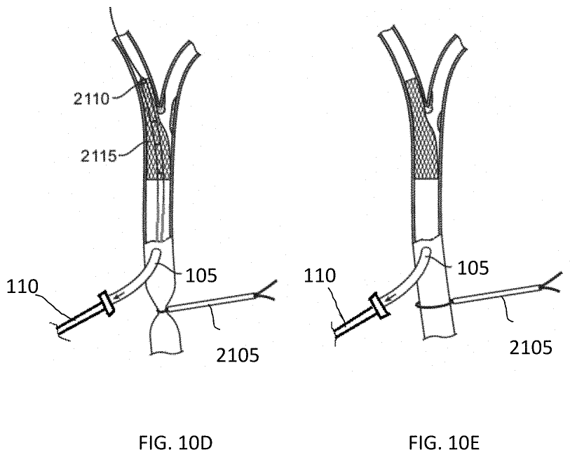

Referring now to FIGS. 10A-10E, flow through the carotid artery bifurcation at different stages of the methods of the present disclosure will be described. A distal sheath 605 of the arterial access device 110 can be introduced into or placed in fluid communication with the common carotid artery CCA via the conduit 105. In some implementations, the distal end of the distal sheath 605 is inserted through the coupler 107 and into the lumen of the conduit 105. The distal end of the distal sheath 605 can insert beyond the distal end of the conduit. Preferably, the distal end of the sheath 605 does not exit the distal end of the conduit 105 to enter the vessel lumen and instead stays within the conduit 105. In other implementations, the arterial access device 110 has no distal sheath 605 and couples directly to the coupler 107 without any tubular feature extending into to lumen of the conduit 105. After the arterial access device 110 has been placed in fluid communication with the common carotid artery CCA through the conduit 105, the blood flow will continue in antegrade direction AG with flow from the common carotid artery entering both the internal carotid artery ICA and the external carotid artery ECA, as shown in FIG. 10A. In still further implementations, no arterial access device 110 is incorporated within the system. The distal end of the conduit 105 is directly coupled to the vessel and the proximal end of the conduit 105 is attached to the reverse flow circuit such as via the coupler 107. This implementation maximizes lumen size through the entire circuit.

The venous return device 115 is then inserted into a venous return site, such as the internal jugular vein IJV (not shown in FIGS. 10A-10E) or femoral vein. The shunt 120 is used to connect the flow lines 615 and 915 of the arterial access device 110 (or the coupler 107 of the conduit 105 in the implementation without an arterial access device 110) and the venous return device 115, respectively (as shown in FIG. 1A). In this manner, the shunt 120 provides a passageway for retrograde flow from the conduit 105, the atrial access device 110, if present, to the venous return device 115. In another embodiment, the shunt 120 connects to an external receptacle 130 rather than to the venous return device 115, as shown in FIG. 1C.

Once all components of the system are in place and connected, flow through the common carotid artery CCA is stopped, typically by use of a tourniquet 2105 or other external vessel occlusion device to occlude the common carotid artery CCA. At that point retrograde flow RG from the external carotid artery ECA and internal carotid artery ICA will begin and will flow through the conduit 105 (the sheath 605, if present), the flow line 615, if present, the shunt 120, if present, and into the venous return device 115 via the flow line 915. The flow control assembly 125 regulates the retrograde flow as described above. FIG. 10B shows the occurrence of retrograde flow RG. While the retrograde flow is maintained, an interventional tool 2110 can be introduced into the vessel, as shown in FIG. 10C. The tool 2110 can be introduced through the hemostasis valve 615 and the proximal extension 610 (not shown in FIGS. 10A-10E) of the arterial access device 110. The tool 2110 can be introduced through the coupler 107 of the conduit 105 and directly into the vessel without being inserted through the arterial access device 110. FIG. 10D shows the interventional tool 2110 is a stent delivery catheter advanced into the internal carotid artery ICA and a stent 2115 deployed at the bifurcation B. The interventional tool can also be an angioplasty catheter, an aspiration catheter, or any of a variety of interventional devices for treating the carotid artery, the internal carotid artery, or any of a variety of cerebral vessels. The conduit 105 can provide an access port for procedures other than TCAR and neurovascular procedures. For example, the conduit 105 can be attached to the CCA to facilitate the delivery of devices to be directed into the proximal CCA, in the aorta, and elsewhere for the treatment of innominate ostial lesions, delivery of stent grants in the aorta (TEVAR), delivery of valves to the aortic root (TAVR), etc. These procedures can typically involve larger devices that would benefit from entry into a vessel through the conduit as opposed to a conventional 8 Fr access sheath. The conduit provides a safer access that is more efficient and easier to enter the proximal CCA, the innominate artery, aorta, and other locations. The larger size of the direct access into the vessel provides particular advantages for insertion of catheters and instruments that tend to be larger and block flow through conventional size sheaths, which renders retrograde flow embolic protection less effective.

The rate of retrograde flow can be increased during periods of higher risk for emboli generation for example while the stent delivery catheter 2110 is being introduced and optionally while the stent 2115 is being deployed. The rate of retrograde flow can be increased also during placement and expansion of balloons for dilatation prior to or after stent deployment. An atherectomy can also be performed before stenting under retrograde flow. The conduit 105 can improve the fluid dynamics for deployment of such devices by providing a larger inner dimension than a conventional access sheath.

Still further optionally, after the stent 2115 has been expanded, the bifurcation B can be flushed by cycling the retrograde flow between a low flow rate and high flow rate. The region within the carotid arteries where the stent has been deployed or other procedure performed may be flushed with blood prior to reestablishing normal blood flow. In particular, while the common carotid artery remains occluded, a balloon catheter or other occlusion element may be advanced into the internal carotid artery and deployed to fully occlude that artery. The common carotid artery may also be occluded using external occlusion mechanism such as a tourniquet 2105. The same maneuver may also be used to perform a post-deployment stent dilatation, which is typically done currently in self-expanding stent procedures. Flow from the common carotid artery and into the external carotid artery may then be reestablished by temporarily opening the occluding means present in the artery. The resulting flow will thus be able to flush the common carotid artery which saw slow, turbulent, or stagnant flow during carotid artery occlusion into the external carotid artery. In addition, the same balloon may be positioned distally of the stent during reverse flow and forward flow then established by temporarily relieving occlusion of the common carotid artery and flushing. Thus, the flushing action occurs in the stented area to help remove loose or loosely adhering embolic debris in that region.

Optionally, while flow from the common carotid artery continues and the internal carotid artery remains blocked, measures can be taken to further loosen emboli from the treated region. For example, mechanical elements may be used to clean or remove loose or loosely attached plaque or other potentially embolic debris within the stent, thrombolytic or other fluid delivery catheters may be used to clean the area, or other procedures may be performed. For example, treatment of in-stent restenosis using balloons, atherectomy, or more stents can be performed under retrograde flow. In another example, the occlusion balloon catheter may include flow or aspiration lumens or channels which open proximal to the balloon. Saline, thrombolytics, or other fluids may be infused and/or blood and debris aspirated to or from the treated area without the need for an additional device. While the emboli thus released will flow into the external carotid artery, the external carotid artery is generally less sensitive to emboli release than the internal carotid artery. By prophylactically removing potential emboli which remain, when flow to the internal carotid artery is reestablished, the risk of emboli release is even further reduced. The emboli can also be released under retrograde flow so that the emboli flows through the shunt 120 to the venous system, a filter in the shunt 120, or the receptacle 130.

After the bifurcation has been cleared of emboli, the tourniquet 2105 (or the occlusion element 129 on the sheath 605, if applicable) can be released, reestablishing antegrade flow, as shown in FIG. 10E. The sheath 605 can then be removed from the conduit 105 or, if no sheath 605 is used in the system, the proximal coupler 660 uncoupled from the coupler 107 on the conduit 105. The conduit 105 can be removed from the vessel after use such as by removing the sutures attaching the distal end of the conduit 105 to the vessel wall and closing the hole through the vessel wall with sutures or another closure method. The vessel may be temporarily clamped proximal and distal to the hole in the vessel to stop blood flow while the hole in the vessel is closed. Alternatively, the conduit 105 can be closed such as using a suture closure technique such as via pre-placed/pre-sewn sutures positioned near a distal end region of the conduit 105.

The conduit 105 can provide an access port for other procedures besides stenting. For example, the conduit 105 can be attached to the CCA to facilitate delivery of a device for stent grafting in the aorta (TEVAR) or delivery of valves to the aortic root (TAVR) in combination with reverse flow. The neuroprotection system can be attached directly to the conduit 105 so that overall flow resistance is far lower than it would otherwise be if an access sheath were used to deliver the devices for the procedure.

FIG. 11 illustrates an interrelated implementation of a large bore delivery system 1100 that can be adapted for use with retrograde or reverse flow blood circulation as described elsewhere herein. The large bore delivery system 1100, like the access systems described in detail elsewhere herein, is further configured for use with a larger bore sheath or delivery system, such as for use in certain carotid artery procedures. As mentioned previously, some carotid artery patients or procedures (e.g. procedures on short/deep vessels, patients with disease, dissections, patients having scar tissue, etc.) may require a larger bore sheath or delivery system, and thus a larger entry point into the carotid artery. Such patients and procedures would benefit from use of a large bore conduit (e.g. as part of a large bore delivery system 1100).

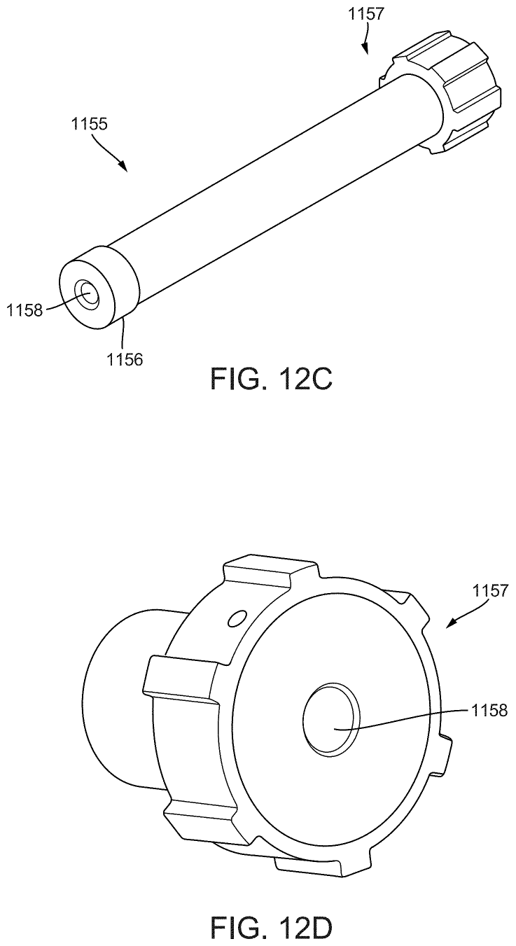

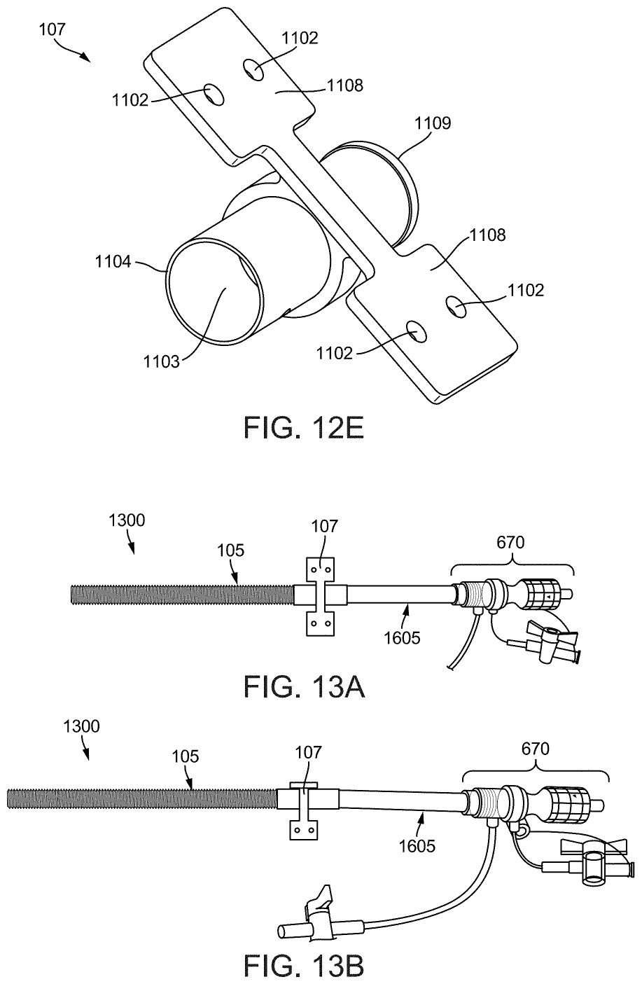

An embodiment of a large bore delivery system 1100 is shown in FIGS. 11-12E. The large bore delivery system 1100 includes a conduit 105, which can be a polymer, fabric, graft, or other material, as described elsewhere herein. The conduit 105 can be coupled at a proximal end to a distal tubular body 1605 via a coupler 107. The distal tubular body 1605 may be a distal sheath 605 as described in further detail elsewhere herein, or may be a length of tubing which may include polymer material or other inorganic material. The length of tubing may be rigid or flexible. The distal tubular body 1605 may include at least a portion of transparent or translucent material, such as to visualize the contents of the distal tubular body 1605. The distal end of the distal tubular body 1605 is coupled to the conduit 105, and at least a portion of a proximal end of the distal tubular body 1605 is inserted into a hub assembly 1635. The hub assembly 1635 may include a hub 1135 with side port 1134, a hemostatic valve 670 such as a Tuohy valve, a Tuohy knob 1140, and a strain relief connector 1130.

Referring now to FIGS. 12A-12E, the components of the large bore delivery system 1100 are illustrated in further detail. As shown in FIG. 12A, a conduit 105 is included, which may include inorganic material or graft material, as described in further detail elsewhere herein. As described previously, use of the large bore delivery system 1100 can improve surgical outcomes, as the tissue surrounding the large bore device does not need to be manipulated to control the access site. The conduit 105 may be coupled to the distal tubular body 1605 via a coupler 107 as described above. The coupler 107 may be a through-type tubing coupler, as shown in FIGS. 12A and 12E. Alternatively, the coupler 107 may be any suitable coupler or connector (e.g. threaded or non-threaded couplers, genderless couplers, flanged couplers, clips, clamps, etc.). The coupler 107 may be constructed of any suitable material, such as a metal, a plastic, a polymer, a bio-absorbable material, a combination of the foregoing, etc. The conduit 105 can also offer increased certainty in the integrity of the material being used for the surgical site closure (i.e. conduit/graft material having more structural integrity than patients' tissue).

FIG. 12A shows an exploded view of the large bore delivery system 1100 including the conduit 105, a coupler 107, and distal tubular body 1605, which may be connected as described above. FIG. 12A further shows an exploded view of the hub assembly 1635. The hub assembly 1635 may include a strain relief connector 1130, a hub 1135 with side port 1134, a hemostatic valve 670 such as a silicone Tuohy valve and a Tuohy knob 1140, and a small bore adapter 1155. The small bore adapter 1155 is configured to allow connection of the large bore delivery system 1100 to a small bore sheath or device. The coupler 107 may be configured as a conduit/polymer tubing connector as shown in FIG. 12A. The coupler 107 provides a seamless transition between the conduit 105 and the distal tubular body 1605. FIG. 12E is a closer view of the coupler 107, configured as a through-type tubing coupler. As shown in FIG. 12E, the coupler 107 has a distal end 1104 and a proximal end 1109. The coupler 107 can have suture tabs 1108 located at a point between the distal end 1104 and the proximal end 1109. The coupler 107 has a bore 1103 having an inner lumen which places the conduit 105 in fluid connection with the distal tubular body 1605 when they are coupled via the coupler 107. The suture tabs 1108 are perpendicular to the bore 1103, and further include holes 1102 for connection of the suture tabs 1108 with the vessel.

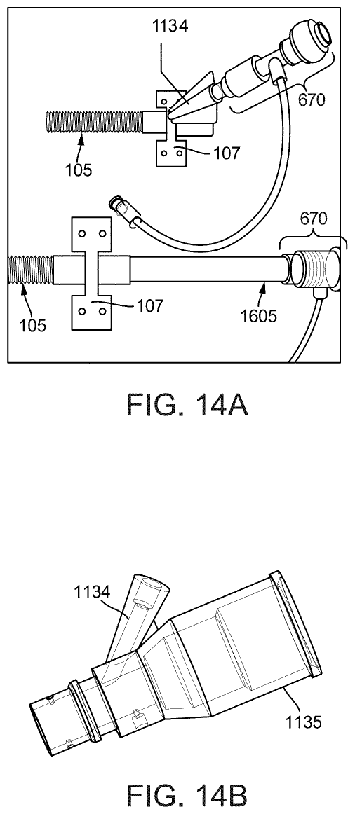

In some instances, such as that illustrated in FIG. 14A, the coupler 107 may also include a side port 1134 for small bore access. For example, a side port 1134 for small bore access may provide greater utility of the access site, which includes a point for reverse flow. A seamless conduit 105 to distal tubular body 1605 transition allows for customization of the overall length of the distal tubular body 1605, such as using less expensive materials (e.g. polymer). The distal tubular body 1605 may also facilitate improved attachment to a hub 1135 or hemostasis valve 1670. The suture tabs may provide a secondary location to stabilize the conduit 105 assembly. The coupler 107 can provide a transition from the conduit 105 to the distal tubular body 1605 or to a hemostasis valve 1670. The coupler 107 may include a side port 1134, for example for small bore adaptor 1155 access (e.g. a 4-8F sheath, a pigtail catheter, or other device) with a hub 1135 and a hemostatic valve or Tuohy valve 1670. The side port 1134 may also be used for reverse flow or for providing distal occlusion. As mentioned previously, the coupler 107 may include suture tabs 1108 for securement of the large bore delivery system or assembly 1100 to the vessel.

The distal tubular body 1605, which may include for example a soft polymer, provides an alternative site for placement of primary or secondary clamps, e.g. pinch clamps, to control blood flow to the large bore delivery system 1100, such as to initiate retrograde flow as described elsewhere herein. The distal tubular body 1605 can include a plastic or polymer which acts as an improved substrate for adhesive bonding to a hemostatic valve 670 or hub 1135. For example, the distal tubular body 1605 provides a length of tubing that is attached, such as via adhesive bonding, to the hemostatic valve 670 or hub 1135, and an access device 110 can be inserted into the distal tubular body 1605 rather than attaching the access device 110 directly to the hemostatic valve 670 or hub 1135. The distal tubular body 1605 can be formed of a soft polymer, and may include a primary and/or a secondary clamp as described above, such as for example a pinch, tube, or ratcheting clamp, used to control blood flow to the large bore delivery system 1100. The distal tubular body 1605 may be lined with a secondary polymer, e.g. PTFE-lined, to provide improved lubricity, or may be configured with a secondary reinforcement, such as via braid or coil, for improved kink resistance. The secondary polymer may be any polymer composition suitable to provide improved lubrication. The secondary reinforcement may include a rigid or flexible plastic or polymer, a metal or metal alloy, or a combination of metals and polymers or plastics.

FIG. 12B is a transparent representation of the hub 1135 with side port 1134.

FIG. 12C is a perspective view of the small bore adapter 1155. The small bore adapter 1155 includes a distal end 1156 and a proximal end 1157. The distal end 1156 couples to the large bore delivery system 1100, while the proximal end 1157 couples to the small bore sheath or devices. FIG. 12D is a closer view of the proximal end 1157 of the small bore adapter 1155, including the coupling channel 1158 where the small bore adapter 1155 couples to the small bore sheath or device. For example, an arterial access device 110 may have a diameter smaller than the diameter of the distal tubular body 1605, such that the arterial access device 110 could not be coupled to the distal tubular body 1605. The small bore adapter 1155 has an inner bore which tapers from a smaller diameter at the proximal end 1157 to a larger diameter at the distal end 1156, thus providing fluid communication between the small bore sheath or device and the distal tubular body 1605.

As discussed elsewhere herein, the conduit 105 can be formed of an inorganic material such as polymer or plastic. The conduit 105 can also be formed of graft material that is formed of tissue such as a patient's own tissue. FIGS. 13A-13B, shows an interrelated implementation of a large bore delivery system 1300 that includes a conduit 105 which may be formed of graft material. As described above, the conduit 105 can be coupled to distal tubular body 1605 via a coupler 107. The graft material can be formed of tissue and/or an inorganic material such as a polymer or plastic. The conduit 105 can be formed of polyethylene terephthalate (Dacron), a polymer, or a bio-absorbable material. The conduit 105 may be convoluted on straight graft for attachment to the vessel or structure being accessed by the large bore delivery system 1300. The distal tubular body 1605 is further coupled to a hemostatic valve 670, such as a passive hemostasis valve as shown in FIGS. 13A and 13B. FIG. 13B shows another angle of the large bore delivery system 1300 illustrating the connection of the conduit 105 to the distal tubular body 1605 via the coupler 107.

Referring to FIGS. 14A-14B, embodiments of the coupler 107 are shown. The coupler 107 may be a polymer through-type tubing connector as shown in the bottom of FIG. 14A and also as shown in FIG. 12E, or may be configured as a connector with a side port 1176 as shown in the top of FIG. 14A. The connector with side port 1176 may may be configured as a small bore access port, which allows introduction of a small bore device into the large bore delivery system 1100. The connector with side port 1176 is used to introduce another device, tool, or substance into the large bore delivery system 1100. FIG. 14B shows a transparent, perspective view of a hub 1135 with side port 1134. The hub 1135 with side port 1134 may be configured for large or small bore access.

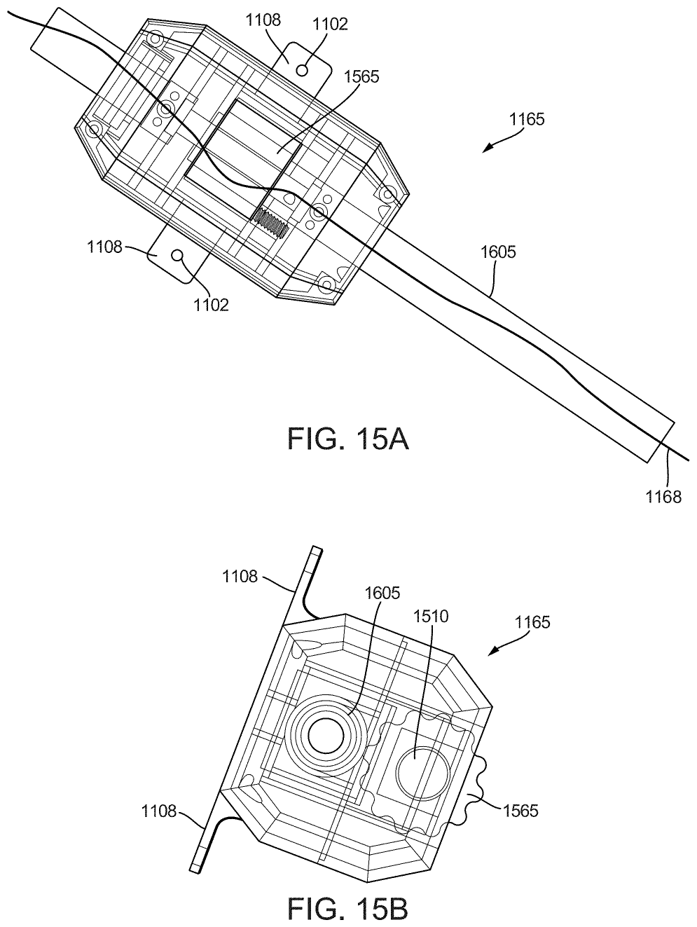

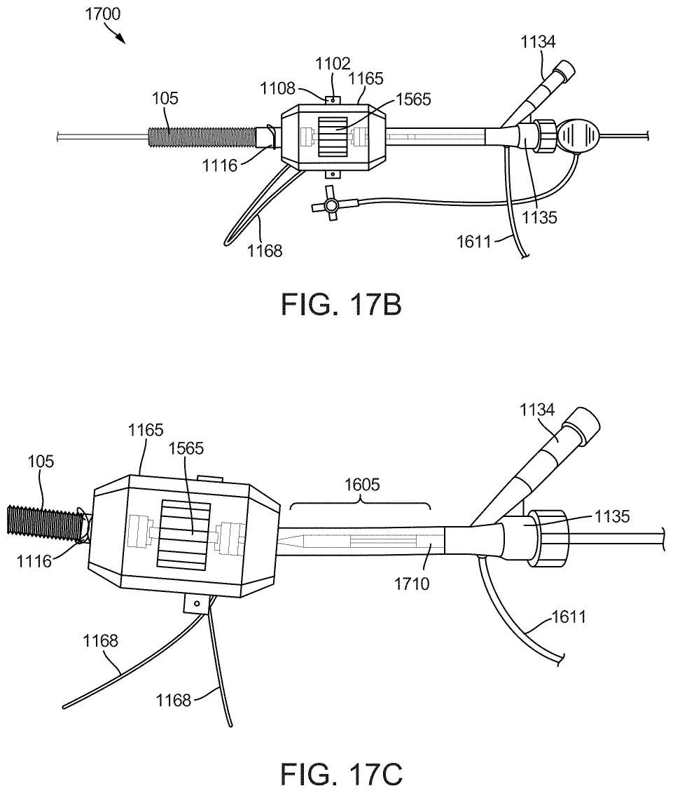

FIGS. 15A-15B and also FIGS. 17A-17C show embodiments of a vessel loop controller 1165. The vessel loop controller 1165 is used to manage vessel loops 1168 (see FIGS. 17A-17C). The vessel loops 1168 can be disposable, single use vessel loops used to occlude, retract, and/or identify vessels, veins, or nerves and tendons, in a variety of medical procedures such as those described herein. In some embodiments, there are one or more vessel loops 1168 are attached to a mechanism 1565 for actuation. For example, the vessel loops 1168 may be wrapped circumferentially around at least a section of distal tubular body 1605. Actuation of the vessel loops 1168 may cinch down the distal tubular body 1605, such as to stop blood flow completely, establish retrograde flow, or to stabilize or “lock in” positioning of a device. The vessel loops 1168 may also be used to control bleeding by sealing completely, or sealing around, devices. In the implementations, a knob such as a Tuohy knob 1140 or other mechanism may be used to actuate the vessel loops 1168 (e.g. a crank, a motor, slides, pulls, etc.). In some implementations, the vessel loops 1168 may be formed of an elastomeric material, such as silicone or the like, that does not undergo elastic deformation under the range of travel of the vessel loops 1168. The vessel loop controller 1165 can be attached to a conduit 105, such as at the location of the coupler 107. The conduit 105 can extend through a bore 1510 in the controller 1165 such that the knob 1140 is located over the conduit 105. The coupler 107 can be configured as a reversible clip or clamp, for reversible attachment of the vessel loop controller 1165 or hub 1135 to the conduit 105 or graft material and place the vessel loop controller 1165 in fluid communication with the conduit 105 or graft material. The vessel loop controller 1165 may then be used to introduce vessel loops 1168 into the large bore delivery system 1100. The coupler 107, when reversible, provides a means to temporarily affix the vessel loop controller 1165 or a hub 1135 to the conduit 105 or graft material.

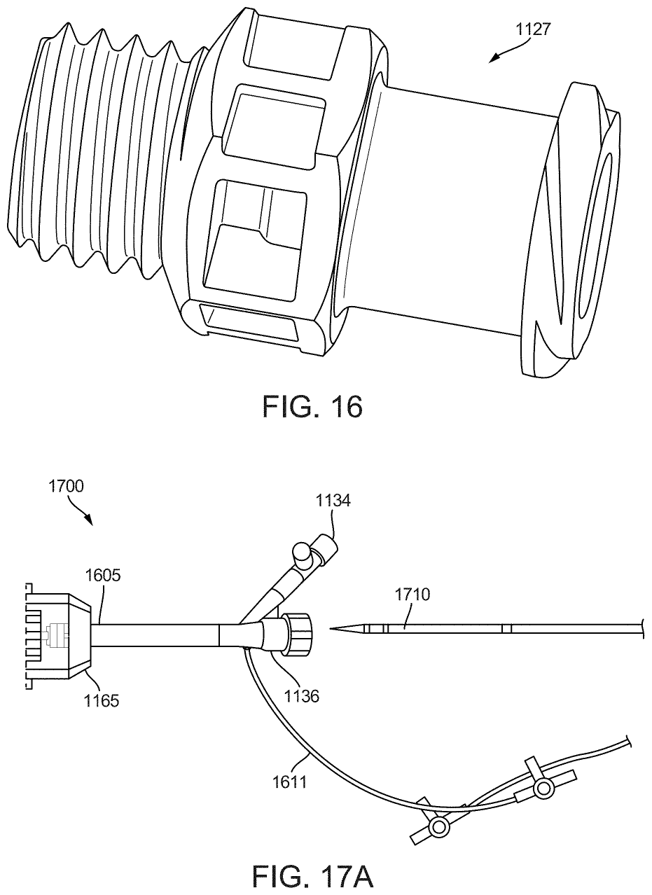

FIG. 16 shows an embodiment of a primary integrated clamp 1127. The primary integrated clamp 1127 is located on the conduit 105 or graft material, and allows a physician to completely stop blood flow during device introduction and removal, and thus minimize blood loss and establish retrograde flow. Moving the location of the clamping from the traditional location at the patient's neck (proximal vessel) to a location on the conduit 105 increases patient comfort and reduces the risk of artery damage or the creation or liberation of embolic material in the event of disease in the artery. In some embodiments, a partially actuated clamp may be used, for example to stabilize the position of the delivery systems within the conduit 105 for therapeutic device delivery. in some embodiments, the primary integrated clamp 1127 may be pre-positioned on the conduit 105 or the distal tubular body 1605, in order to stop blood flow through the conduit 105 assembly during device exchanges. In some embodiments, the primary integrated clamp 1127 may be used to partially clamp the conduit 105 or distal tubular body 1605 to the sheath or delivery system, such as to provide increased stability. The primary integrated clamp 1127 may be configured as a luer fitting, as shown in FIG. 16, or as any other suitable clamp (e.g. pinch clamps, tube clamps, ratcheting clamps, vessel loops, pipe clamps, or the like).

Referring now to FIG. 17A, another embodiment of a large bore delivery system 1700 incorporating the vessel loop controller 1165 described above is shown. The large bore delivery system 1700 sized to accommodate a delivery device 1710, such as a TAVR delivery device. The system 1700 can include a hub 1135 with side port 1134, distal tubular body 1605, and flushing tubing 1611. In some embodiments, the side port 1120 of the hub 1135 with side port 1134 is optional and a conduit hub 1136 is used. The delivery device 1710 is inserted into the proximal end of the hub 1135 with side port 1134. The hub 1135 with side port 1134 is connected at the distal end to the distal tubular body 1605. At a distal point on the hub 1135 with side port 1134, proximal to the distal end of the hub 1135 with side port 1134 is the connection point of the flushing tubing 1611. The flushing tubing 1611 can facilitate removal of liquids or gases from the large bore delivery system 1700. The distal tubular body 1605 may then be connected at its distal end to a hub, such as a vessel loop controller 1165. The conduit hub 1136 may provide a secondary seal or mechanism to provide hemostasis. In some embodiments, the secondary seal or mechanism for hemostasis may be a passive design, such as for example a cross-cut silicone seal or a series of seals. Alternatively, the seal may be an active seal, such as a Tuohy valve or the like. Other possible features include a hub 1135 with side port 1134 or small bore adaptor 1155 for delivery of small bore devices or sheathes, or an insert into the main hemostasis mechanism which allows small bore devices to be delivered.

The distal tubular body 1605 is configured to attach to the vessel loop controller 1165, and may provide a bridge between the conduit hub 1136 and the conduit 105, which may be a graft. In some implementations, the distal tubular body 1605 has a durometer from about 15 Shore A to about 80 Shore A. In implementations, the distal tubular body 1605 may have a durometer from about 10 Shore A to about 100 Shore A. In implementations, the distal tubular body 1605 may have a durometer from about 20 Shore A to about 90 Shore A. In implementations, the distal tubular body 1605 may have a durometer from about 30 Shore A to about 80 Shore A. In implementations, the distal tubular body 1605 may have a durometer from about 40 Shore A to about 70 Shore A. In implementations, the distal tubular body 1605 may have a durometer from about 50 Shore A to about 60 Shore A. In some implementations, the sections of the distal tubular body 1605 where the vessel loops 1168 are located after insertion may have a reduced wall thickness, such as to facilitate actuation of the vessel loops 1168 in these areas. In some implementations, the distal tubular body 1605 may press fit onto the conduit hub 1136. In some implementations, the distal tubular body 1605 may have a clamp or other connector for attachment to the conduit hub 1136 and/or the graft material. In some implementations, an adhesive or thermal bonding agent may also be employed to attached the distal tubular body 1605 to the conduit hub 1136 and/or the graft material. In some implementations, the length of distal tubular body 1605 between the conduit hub 1136 and the vessel loop controller 1165 may be used for removal of air bubbles from an implant, i.e. deairing, prior to delivery of the implant. This design may eliminate loading tubes and other features commonly used with balloon expandable valves and other traditional interventions. This distal tubular body 1605 region also allows a user to pull the delivery devices 1710 and/or sheath(es) into the distal tubular body 1605 region, and subsequently to close the vessel loops fully, and to remove the delivery device(s) 1710 and/or sheath(es) in order to prevent blood loss during removal.

FIG. 17B shows the large bore delivery system 1700 including the conduit 105 and clamp 1116, vessel loop controller 1165, hub 1135 with side port 1134, and distal tubular body 1605. The conduit 105 can be formed of Dacron, or other suitable materials as described elsewhere herein. The clamp 1116 is configured to couple the conduit 105 to the vessel loop controller 1165. The clamp 1116 may be any clip or clamp, permanent or reversible, suitable for connecting the conduit to the vessel loop controller 1165. FIG. 17C is a closer view of the distal tubular body 1605 with a delivery device 1710 inserted therein. A portion of the distal tubular body 1605 may act as a window such that a user can visualize the delivery device 1710 within the distal tubular body 1605. Additionally, when the delivery system is positioned within the portion of the flexible tubing forming the window 1610, the user may perform blood flow control or remove air from the large bore delivery system 1700.

In various implementations, description is made with reference to the figures. However, certain implementations may be practiced without one or more of these specific details, or in combination with other known methods and configurations. In the description, numerous specific details are set forth, such as specific configurations, dimensions, and processes, in order to provide a thorough understanding of the implementations. In other instances, well-known processes and manufacturing techniques have not been described in particular detail in order to not unnecessarily obscure the description. Reference throughout this specification to “one embodiment,” “an embodiment,” “one implementation, “an implementation,” or the like, means that a particular feature, structure, configuration, or characteristic described is included in at least one embodiment or implementation. Thus, the appearance of the phrase “one embodiment,” “an embodiment,” “one implementation, “an implementation,” or the like, in various places throughout this specification are not necessarily referring to the same embodiment or implementation. Furthermore, the particular features, structures, configurations, or characteristics may be combined in any suitable manner in one or more implementations.

The use of relative terms throughout the description may denote a relative position or direction. For example, “distal” may indicate a first direction away from a reference point. Similarly, “proximal” may indicate a location in a second direction opposite to the first direction. However, such terms are provided to establish relative frames of reference, and are not intended to limit the use or orientation of the catheters and/or delivery systems to a specific configuration described in the various implementations.

The word “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

1-19. (canceled)

20. A system for accessing an artery via a trans-carotid approach, comprising:
a conduit having an inner lumen extending between a proximal end and a distal end, the distal end of the conduit configured for surgical end-to-side colligation with a vessel;
a coupler positioned at the proximal end of the conduit; and
a shunt fluidly connected to the coupler so that the inner lumen of the conduit is coupled to a lumen of the shunt that provides a pathway for blood to flow from the conduit through the shunt and to a return site.

21. The system of claim 20, further comprising a port fluidly communicating with the shunt, wherein the port is configured to connect an aspiration device to the shunt, wherein the distal connector of the arterial access device is configured to operatively couple with the coupler at the proximal end of the conduit placing the lumen of the arterial access device in fluid communication with the inner lumen of the conduit.

22. The system of claim 20, wherein the conduit is a flexible, tubular structure formed of a biotextile.

23. The system of claim 20, wherein the conduit is a vascular graft.

24. The system of claim 23, wherein the conduit provides an access port for a procedure in an innominate ostia, aorta, aortic root, carotid artery, or a cerebral vessel.

25. A method for treating a patient, the method comprising:
attaching a conduit to a wall of a vessel, the conduit having an inner lumen extending between a proximal end and a distal end, the distal end of the conduit attached to the wall;
forming an arteriotomy in the wall of the vessel;
inserting a device through the inner lumen of the conduit and into the vessel; and
performing a treatment with the device.

26. The method of claim 25, wherein the proximal end of the conduit comprises a coupler, wherein the device is inserted through the coupler into the inner lumen of the conduit.

27. The method of claim 25, wherein attaching the conduit to the wall further comprises suturing the conduit to the wall with sutures.

28. The method of claim 27, further comprising tightening the sutures for primary closure of the vessel following performing the treatment.

29. The method of claim 25, wherein the distal end of the conduit comprises a mechanical element to facilitate attaching the conduit to the vessel.

30. The method of claim 29, wherein the mechanical element comprises a hood, gasket, or suture ring.

31. The method of claim 25, wherein forming the arteriotomy comprises forming the arteriotomy through the inner lumen of the conduit attached to the wall of the vessel.

32. The method of claim 25, further comprising reversing blood flow through the vessel while performing the treatment.

33. The method of claim 32, wherein the vessel comprises a common carotid artery.

34. The method of claim 33, further comprising advancing the device through the common carotid artery to an innominate artery, aortic arch, descending aorta, ascending aorta, aortic root, coronary artery, internal carotid artery, external carotid artery, or an intracranial vessel.

35. The method of claim 34, wherein the device comprises a balloon catheter, a stent delivery catheter, or an aspiration catheter.

36. The method of claim 25, wherein the treatment comprises one or more of delivery of a stent, angioplasty dilation, delivery of a stent graft, delivery of a valve, aspiration embolectomy and combinations thereof.

37. The method of claim 25, wherein the diameter of the inner lumen of the conduit is between about 6 mm and about 16 mm.

38. The method of claim 25, wherein the diameter of the inner lumen of the conduit is between about 8 mm and about 10 mm.

39. A method for treating a patient having an atypical anatomy, the method comprising:
attaching a conduit to a wall of a vessel, the conduit having an inner lumen extending between a proximal end and a distal end, the distal end of the conduit attached to the wall;
forming an arteriotomy in the wall of the vessel;
inserting a device through the inner lumen of the conduit and into the vessel; and
performing a treatment with the device.

40-53. (canceled)
